(19) United States
(12) Patent Application Publication
(10) Pub. No.: US 2022/0218831 A1
(43) Pub. Date: Jul. 14, 2022

(54) DEGRADATION OF AURORA KINASE (AURK) BY CONJUGATION OF AURK INHIBITORS WITH E3 LIGASE LIGAND

(71) Applicant: Nemucore Medical Innovations, Inc., Wellesley, MA (US)

(72) Inventor: Timothy Patrick COLEMAN, Wellesley, MA (US)

(21) Appl. No.: 17/607,586

(22) PCT Filed: Apr. 30, 2020
(86) PCT No.: PCT/US20/30663

§ 371 Date: Oct. 29, 2021

Related U.S. Application Data
(60) Provisional application No. 62/841,365, filed on May 1, 2019.

(51) Int. Cl. A61K 47/54; A61P 35/00

(52) U.S. Cl. CPC A61K 47/556; A61P 35/00; A61K 47/545

(57) ABSTRACT

Provided herein are compounds, which act as protein degradation inducing moieties for aurora kinase (AURK). The present application also relates to methods for the targeted degradation of AURK through the use of the compounds that link a ubiquitin ligase-binding moiety to a ligand that is capable of binding to AURK which can be utilized in the treatment of disorders modulated by AURK.

### RELATED APPLICATION

This application claims priority to U.S. Provisional Application No. 62/841,365 filed on May 1, 2019, the content of which is incorporated herein in its entirety.

### BACKGROUND

Since its discovery in 1997, the mammalian Aurora family of serine/threonine kinases has been closely linked to tumorigenesis. The three known mammalian family members, Aurora Kinase-A (AURKA), -B (AURKB) and -C (AURKC), are highly homologous proteins responsible for chromosome segregation, mitotic spindle function and cytokinesis. Aurora expression is low or undetectable in resting cells, with expression and activity peaking during the G2 and mitotic phases in cycling cells. In mammalian cells, proposed substrates for the Aurora A and B kinases include histone H3, CENP-A, myosin II regulatory light chain, protein phosphatase 1, TPX2, INCENP, p53 and survivin, many of which are required for cell division. The Aurora kinases have been reported to be overexpressed in a wide range of human tumors. Elevated expression of AURKA has been detected in colorectal, ovarian and pancreatic cancers and in invasive duct adenocarcinomas of the breast. High levels of AURKA have also been reported in renal, cervical, neuroblastoma, melanoma, lymphoma, pancreatic and prostate tumor cell lines. Amplification/over-expression of AURKA is observed in human bladder cancers and amplification of AURKA is associated with aneuploidy and aggressive clinical behavior. Moreover, amplification of the AURKA locus correlates with poor prognosis for patients with node-negative breast cancer. In addition, an allelic variant, isoleucine at amino acid position 31, is reported to be a low-penetrance tumor-susceptibility gene and displays greater transforming potential than the phenylalanine-31 variant and is associated with increased risk for advanced and metastatic disease. Like AURKA, AURKB is also highly expressed in multiple human tumor cell lines, including leukemic cells. Levels of AURKB increase as a function of Duke's stage in primary colorectal cancers. AURKC, which is normally only found in germ cells, is also overexpressed in a high percentage of primary colorectal cancers and in a variety of tumor cell lines including cervical adenocarcinoma and breast carcinoma cells.

Therefore, there remains a need for structurally diverse inhibitors of Aurora kinase activity, particularly ones that are potent and/or selective inhibitors of AURKA and/or AURKB.

### SUMMARY

Provided herein are compounds that function to recruit targeted proteins to E3 ubiquitin ligase for degradation, and methods of preparation and uses thereof.

The compounds provided herein, e.g., compounds of Formula X, target proteins, such as AURKA, AURKB, and/or AURKC for degradation. Targeting these proteins for degradation provides numerous advantages over merely inhibiting protein function (e.g., kinase activity). By targeting proteins, such as Aurora kinases, for degradation, small molecules of Formula X can a) overcome resistance in certain cases; b) prolong the kinetics of drug effect by destroying the protein, thus requiring resynthesis of the protein even after the compound has been metabolized; c) target all functions of a protein at once rather than a specific catalytic activity or binding event; d) expand the number of drug targets by including all proteins that a ligand can be developed for, rather than proteins whose activity (e.g., kinase activity) can be affected by a small molecule inhibitor, antagonist or agonist; and e) have increased potency compared to inhibitors due to the possibility of the small molecule acting catalytically.

In an aspect, provided herein are compounds of Formula X:

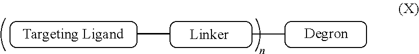

wherein:

the targeting ligand is capable of binding to a targeted protein, such as an aurora kinase (e.g., AURKA, AURKB, or AURKC);

the linker is a group that covalently binds to the targeting ligand and the degron;

the degron is capable of binding to a ubiquitin ligase, such as an E3 ubiquitin ligase (e.g., cereblon); and

n is 1-3.

In an embodiment, n is 1. In another embodiment n is 2. In another embodiment, n is 3.

In another aspect, provided herein are methods of targeting proteins for degradation through the use of compounds, including compounds that link an E3 ubiquitin ligase-binding moiety to a ligand that binds the targeted proteins.

In an embodiment of Formula X, the targeting ligand is a compound of Formula I:

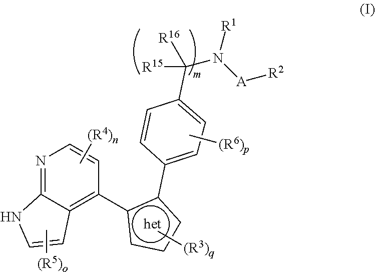

or a pharmaceutically acceptable salt thereof;

wherein:

R¹ is H or C₁-C₆ alkyl;

R² is covalently bonded to the linker and is selected from the group consisting of —NH—, —NCH₃—, —O—, —S—, and —CH₂—;

R³ is selected from the group consisting of C₁-C₆ alkyl, C₁-C₆ alkoxy, hydroxy, amino, and halo;

R⁴ is selected from the group consisting of C₁-C₆ alkyl, C₁-C₆ alkoxy, hydroxy, amino, and halo;

R⁵ is selected from the group consisting of C₁-C₆ alkyl, C₁-C₆ alkoxy, C₅-C₁ heteroaryl, C₆-C₁ aryl, C₃-C₆ cycloalkyl, and C₃-C₆ heterocycloalkyl, wherein C₅-C₁ heteroaryl and C₆-C₁ aryl are optionally substituted with C₁-C₆ alkyl, C₁-C₆ alkoxy, and C₁-C₆ alkylamine;

R⁶ is selected from the group consisting of C₁-C₆ alkyl, C₁-C₆ alkoxy, hydroxy, amino, and halo;

R¹⁵ is selected from the group consisting of C₁-C₆ alkyl, C₁-C₆ alkoxy, hydroxy, amino, and halo;

R¹⁶ is selected from the group consisting of C₁-C₆ alkyl, C₁-C₆ alkoxy, hydroxy, amino, and halo;

het is C₅-C₁ₐ heteroaryl or C₃-C₆ heterocycloalkyl;

A is selected from the group consisting of absent, —C(O)—, —CH₂—, —SO₂—, and —O—;

m is 0, 1, 2, 3, or 4;

n is 0, 1, or 2;

o is 0, 1, or 2;

p is 0, 1, 2, 3, or 4; and

q is 0, 1, 2, or 3.

In another embodiment, the compound of Formula X is a compound of Formula II:

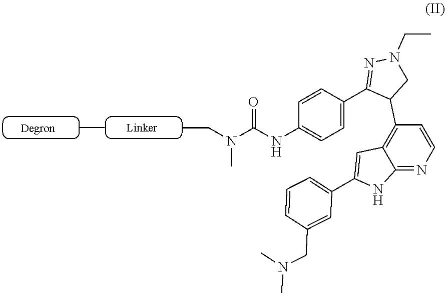

or a pharmaceutically acceptable salt thereof;

wherein the targeting ligand is covalently bonded to the linker.

In yet another embodiment, the compound of Formula X is a compound of Formula D1:

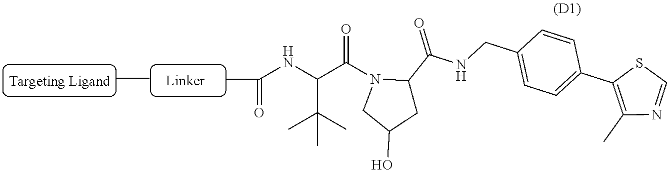

or a stereoisomer thereof;

wherein the degron is covalently bonded to the linker.

In still another embodiment, the compound of Formula X is a compound of Formula D2:

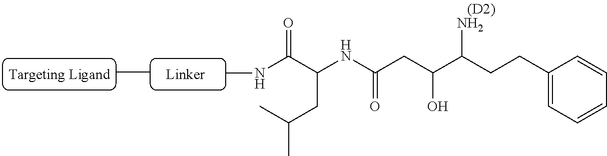

or a stereoisomer thereof;

wherein the degron is covalently bonded to the linker.

In an embodiment, the compound of Formula X is a compound of Formula D3:

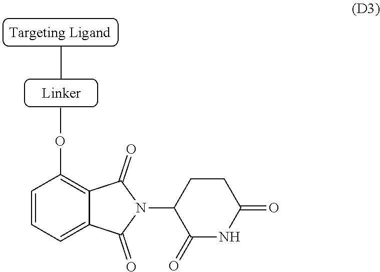

or a stereoisomer thereof;

wherein the degron is covalently bonded to the linker.

In an embodiment, the compound of Formula X is a compound of Formula D4:

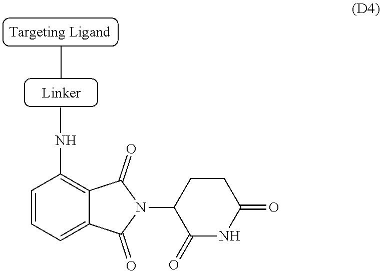

or a stereoisomer thereof;

wherein the degron is covalently bonded to the linker.

In an embodiment, the compound of Formula X is a compound of Formula D5:

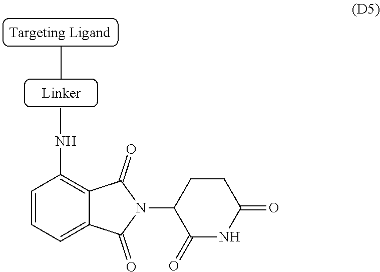

or a stereoisomer thereof;

wherein the degron is covalently bonded to the linker.

In an embodiment, the compound of Formula X is a compound of Formula D6:

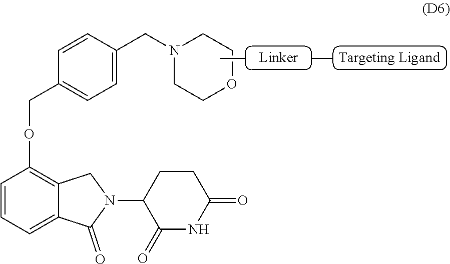

or a stereoisomer thereof;

wherein the degron is covalently bonded to the linker.

In yet another embodiment, the compound of Formula X is a compound of Formula D7:

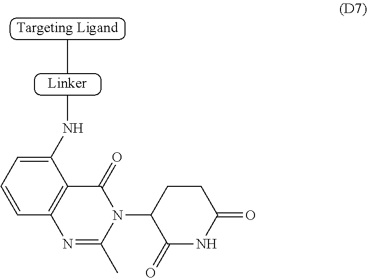

or a stereoisomer thereof;

wherein the degron is covalently bonded to the linker.

In an embodiment, the linker is C₁-C₆ alkyl and the linker is covalently bonded to the degron and covalently bonded to the targeting ligand.

In another embodiment, the linker is selected from the group consisting of:

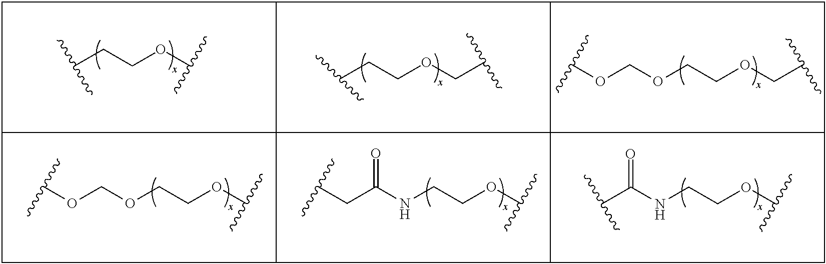

wherein x is 0, 1, 2, 3, 4, or 5; and

the linker is covalently bonded to the degron and covalently bonded to the targeting ligand.

In yet another embodiment, the compound of Formula X is a compound of Formula L1:

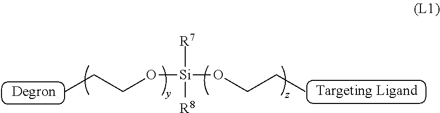

wherein:

R⁷ and R³ are independently selected from the group consisting of methyl, ethyl, propyl, isopropyl, and tert-butyl;

y is 1, 2, 3, 4, or 5;

z is 1, 2, 3, 4, or 5; and

the linker is covalently bonded to the degron and covalently bonded to the targeting ligand.

In a preferred embodiment, the compound of Formula X is selected from the group consisting of:

TABLE 1

| Com- | |
|---|---|
| pound | |
| No. | Structure |
| 1 | 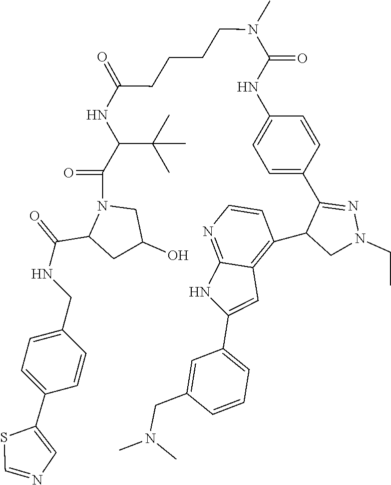 [SMILES: CCN1CC(c2ccnc3[nH]c(-c4cccc(CN(C)C)c4)cc23)C(c2ccc(NC(=O)N(C)CCCCC(=O)NC(C(=O)N3CC(O)CC3C(=O)NCc3ccc(-c4cncs4)cc3)C(C)(C)C)cc2)=N1] |
| 2 | 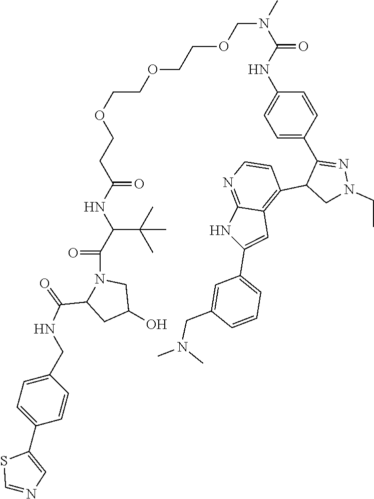 [SMILES: CCN1CC(c2ccnc3[nH]c(-c4cccc(CN(C)C)c4)cc23)C(c2ccc(NC(=O)N(C)COCCOCCOCCC(=O)NC(C(=O)N3CC(O)CC3C(=O)NCc3ccc(-c4cncs4)cc3)C(C)(C)C)cc2)=N1] |
| 3 | 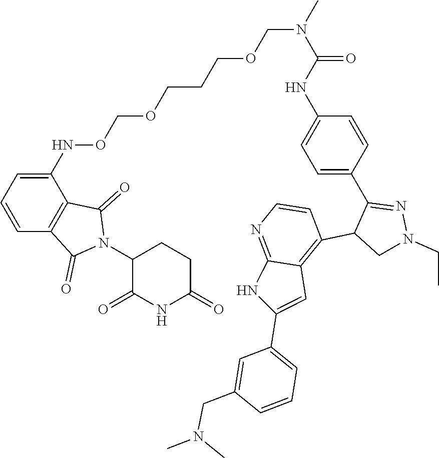 [SMILES: CCN1CC(c2ccnc3[nH]c(-c4cccc(CN(C)C)c4)cc23)C(c2ccc(NC(=O)N(C)COCCCOCONc3cccc4c3C(=O)N(C3CCC(=O)NC3=O)C4=O)cc2)=N1] |
| 4 | 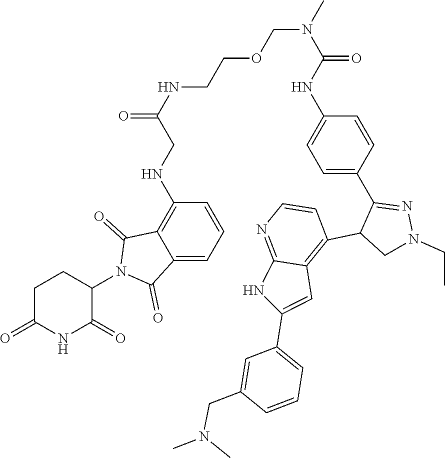 [SMILES: CCN1CC(c2ccnc3[nH]c(-c4cccc(CN(C)C)c4)cc23)C(c2ccc(NC(=O)N(C)COCCNC(=O)CNc3cccc4c3C(=O)N(C3CCC(=O)NC3=O)C4=O)cc2)=N1] |
| 5 | 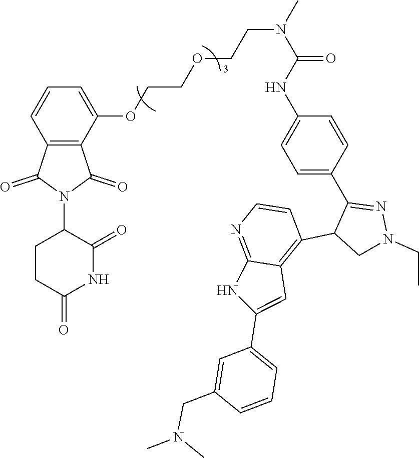 |
| 6 | 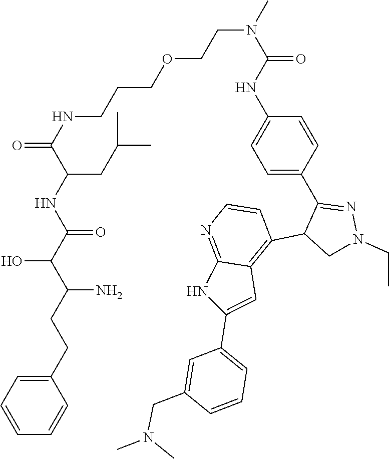 [SMILES: CCN1CC(c2ccnc3[nH]c(-c4cccc(CN(C)C)c4)cc23)C(c2ccc(NC(=O)N(C)CCOCCCNC(=O)C(CC(C)C)NC(=O)C(O)C(N)CCc3ccccc3)cc2)=N1] |
| 7 | 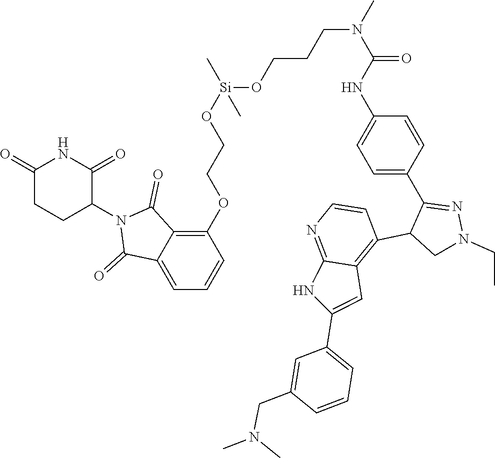 [SMILES: CCN1CC(c2ccnc3[nH]c(-c4cccc(CN(C)C)c4)cc23)C(c2ccc(NC(=O)N(C)CCCO[Si](C)(C)OCCOc3cccc4c3C(=O)N(C3CCC(=O)NC3=O)C4=O)cc2)=N1] |
| 8 | 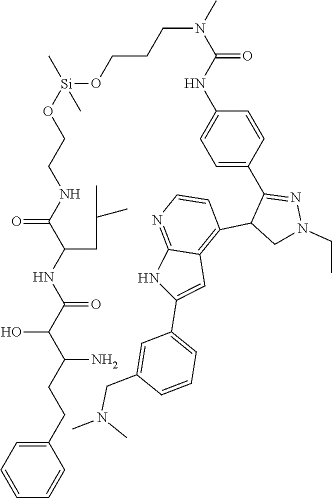 [SMILES: CCN1CC(c2ccnc3[nH]c(-c4cccc(CN(C)C)c4)cc23)C(c2ccc(NC(=O)N(C)CCCO[Si](C)(C)OCCNC(=O)C(CC(C)C)NC(=O)C(O)C(N)CCc3ccccc3)cc2)=N1] |

or pharmaceutically acceptable salts thereof.

In another aspect, provided herein are methods of inhibiting a kinase in a subject in need thereof, comprising administering to the subject an effective amount of a compound of Formula X, or a pharmaceutically acceptable salt thereof, or a pharmaceutical composition comprising a compound of Formula X, or a pharmaceutically acceptable salt thereof.

In an embodiment, the kinase is AURKA. In another embodiment, the kinase is AURKB. In yet another embodiment, the kinase is AURKC.

In yet another aspect, provided herein are methods of modulating (e.g., decreasing) the amount of a kinase in a subject in need thereof, comprising administering to the subject a therapeutically effective amount of a compound of Formula X, or a pharmaceutically acceptable salt thereof, or a pharmaceutical composition comprising a compound of Formula X, or a pharmaceutically acceptable salt thereof.

In still another aspect, provided herein are methods of treating or preventing a disease in a subject in need thereof, comprising administering to the subject an effective amount of a compound of Formula X or a pharmaceutically acceptable salt thereof, or a pharmaceutical composition comprising a compound of Formula X, or a pharmaceutically acceptable salt thereof. In another embodiment, the disease is a proliferative disease.

In an aspect, provided herein are methods of treating cancer in a subject in need thereof, comprising administering to the subject an effective amount of a compound of Formula X or a pharmaceutically acceptable salt thereof, or a pharmaceutical composition comprising a compound of Formula X, or a pharmaceutically acceptable salt thereof. In another embodiment, the subject is identified as being in need of AURKA, AURKB, and/or AURKC inhibition for the treatment of cancer.

In an embodiment, the cancer is selected from the group consisting of non-small cell lung carcinoma (NSCLC), mesothelioma, glioblastoma, oral cancer, malignant endometrium, hepatocellular carcinoma, testicular germ cell tumors, ovarian cancer, thyroid cancer, colon cancer, and prostate cancer.

In another embodiment, the cancer is selected from the group consisting of acute myeloid leukemia (AML), acute lymphocytic leukemia (ALL), and chronic lymphocytic leukemia (CLL).

In another aspect, provided herein are kits comprising a compound of Formula X capable of inhibiting kinase activity. In an embodiment, the kinase is Aurora kinase-A (AURKA). In another embodiment, the kinase is Aurora kinase-B (AURKB). In an embodiment, the kinase is Aurora kinase-C (AURKC).

In yet another aspect, provided herein are kits comprising a compound of Formula X capable of modulating (e.g., decreasing) the amount of AURKA, AURKB, and/or AURKC.

In an aspect, provided herein are kits for the treatment of a disease, wherein the kit comprises a compound of Formula X. In an embodiment, the disease is a kinase-mediated disease. In another embodiment, the kinase is Aurora kinase-A (AURKA). In yet another embodiment, the kinase is Aurora kinase-B (AURKB). In another embodiment, the kinase is Aurora kinase-C (AURKC). In still another embodiment, the disease is a proliferative disease. In an embodiment, the proliferative disease is cancer.

In an aspect, provided herein are compounds of Formula X, or a pharmaceutically acceptable salt thereof, or a pharmaceutical composition comprising a compound of Formula X, or a pharmaceutically acceptable salt thereof, for use in the manufacture of a medicament for inhibiting a kinase or for modulating (e.g., decreasing) the amount of a kinase. In an embodiment, the kinase is AURKA. In another embodiment, the kinase is AURKB. In another embodiment, the kinase is AURKC.

In another aspect, provided herein are compounds of Formula X, or a pharmaceutically acceptable salt thereof, or a pharmaceutical composition comprising a compound of Formula X, or a pharmaceutically acceptable salt thereof, for use in the manufacture of a medicament for treating or preventing a disease. In an embodiment, the disease is a kinase-mediated disorder. In another embodiment, the disease is a proliferative disease. In another embodiment, the disease is mediated by AURKA, AURKB, and/or AURKC.

In yet another aspect, provided herein are compounds of Formula X, or a pharmaceutically acceptable salt thereof, or a pharmaceutical composition comprising a compound of Formula X, or a pharmaceutically acceptable salt thereof, for use in the manufacture of a medication for treating or preventing cancer in a subject, wherein the subject is identified as being in need of AURKA, AURKB, and/or AURKC inhibition for the treatment of cancer.

In still another aspect, provided herein are compounds of Formula X, or a pharmaceutically acceptable salt thereof, or a pharmaceutical composition of the application, for use in inhibiting a kinase or modulating the amount of a kinase. In an embodiment, the kinase is AURKA. In another embodiment, the kinase is AURKB. In yet another embodiment, the kinase is AURKC.

In an aspect, provided herein are compounds of Formula X, or a pharmaceutically acceptable salt thereof, or a pharmaceutical composition comprising a compound of Formula X, or a pharmaceutically acceptable salt thereof, for use in treating or preventing a disease.

In an embodiment, the disease is a kinase-mediated disorder. In another embodiment, the disease is a proliferative disease. In yet another embodiment, the disease is mediated by AURKA. In still another embodiment, the disease is mediated by AURKB. In another embodiment, the disease is mediated by AURKC.

In another aspect, provided herein are compounds of Formula X, or a pharmaceutically acceptable salt thereof, or a pharmaceutical composition comprising a compound of Formula X, or a pharmaceutically acceptable salt thereof, for use in treating cancer in a subject, wherein the subject is identified as being in need of AURKA, AURKB, and/or AURKC inhibition for the treatment or prevention of cancer.

In yet another aspect, the compounds and compositions provided herein have an improved efficacy and/or safety profile relative to known AURKA, AURKB, and AURKC inhibitors. The present application also provides agents with novel mechanisms of action toward Aurora kinases in the treatment of various types of diseases including cancer and metastasis.

The compounds and methods provided herein are useful in the treatment of diseases or disorders in which pathogenic or oncogenic endogenous proteins (e.g., AURKB) play a role, such as cancer.

The contents of all references (including literature references, issued patents, published patent applications, and co-pending patent applications) cited throughout this application are hereby expressly incorporated herein in their entireties by reference. The references cited herein are not admitted to be prior art to the application.

### BRIEF DESCRIPTION OF THE DRAWINGS

FIG. 1 illustrates the anti-tumor activity of the free target ligand of Formula II alone, or in combination with erlotinib for H2122 NSCLC models.

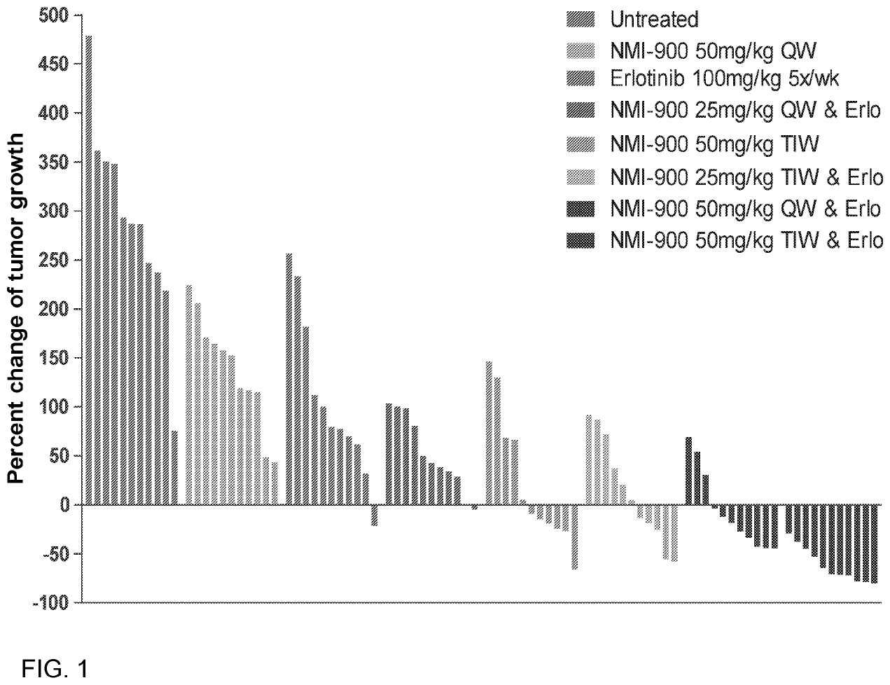

FIG. 2 illustrates the anti-tumor activity of the free target ligand of Formula II alone, or in combination with cytarabine for HL60 AML models.

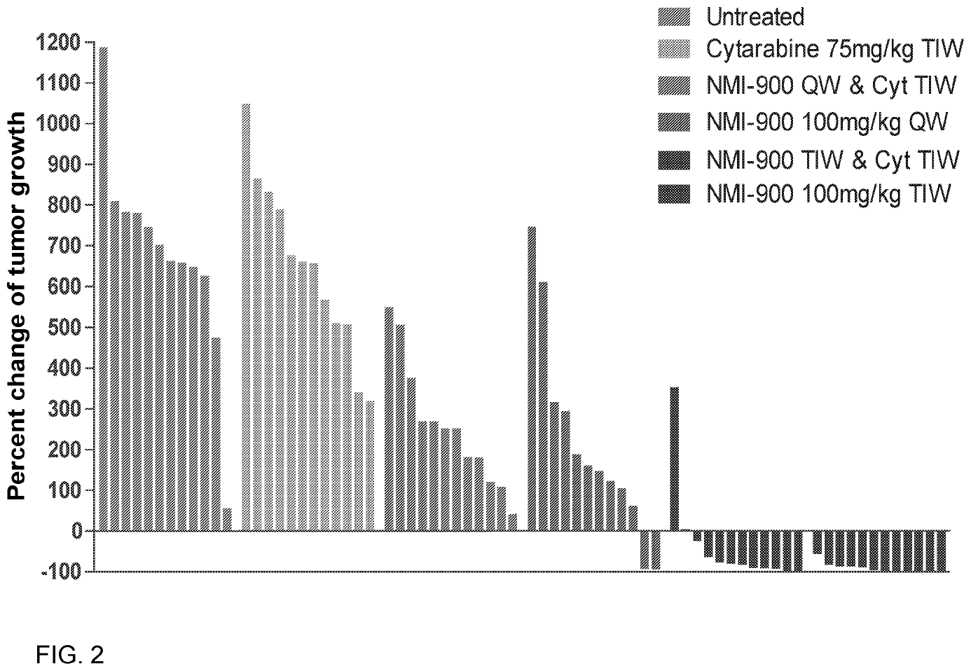

FIG. 3 depicts the synergistic effect observed when the free target ligand of Formula II (GSK-1070916) is combined with JQ1.

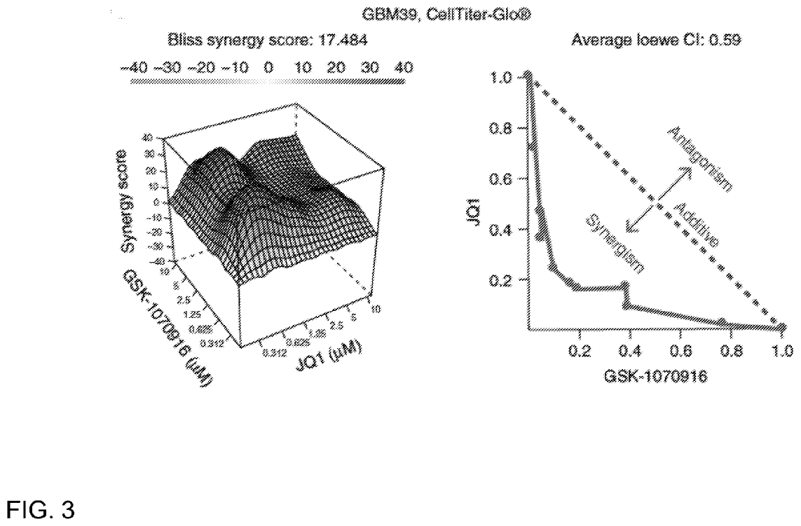

FIG. 4 shows the binding affinity for the free target ligand of Formula II (GSK-1070916) compared to other known Aurora kinase inhibitors.

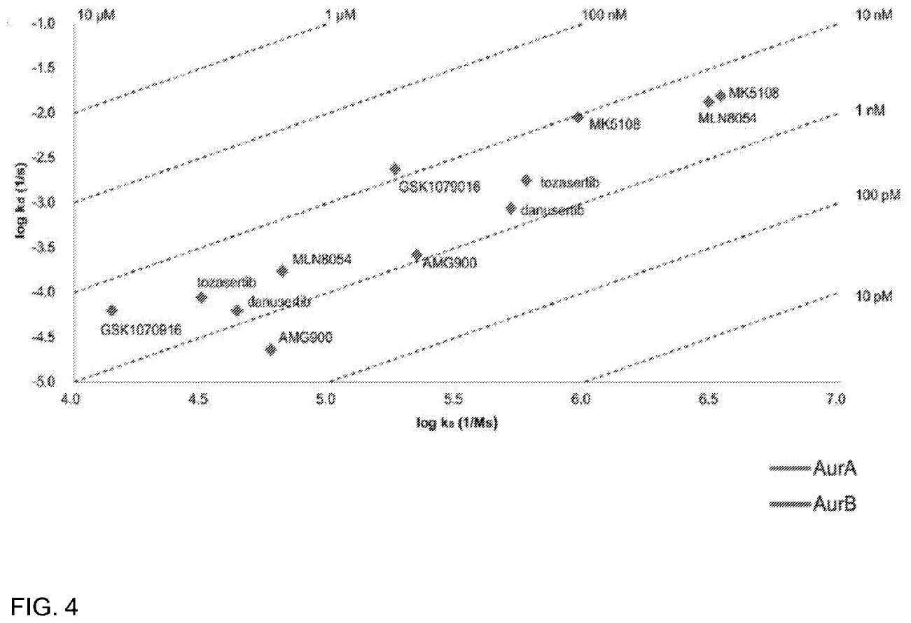

FIG. 5 shows the efficacy of the free target ligand of Formula II (NMI-900) against ovarian carcinoma A2780 xenografts when used as a monotherapy or in combination with cisplatin.

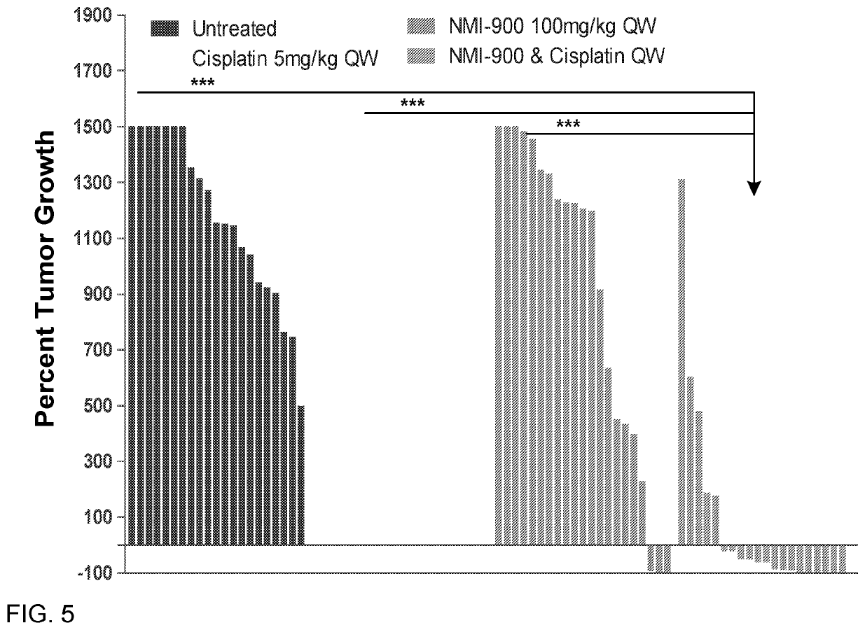

### DETAILED DESCRIPTION

Provided herein are compounds, e.g., compounds of Formula X, or pharmaceutically acceptable salts thereof, that are useful for in the treatment of a disease in a subject in need thereof.

In a non-limiting aspect, these compounds can inhibit kinases. In a particular embodiment, the compounds provided herein are considered AURKA, AURKB, and/or AURKC inhibitors. As such, in one aspect, the compounds provided herein are useful for the treatment of a kinase-mediated disease in a subject in need thereof by acting as a AURKA, AURKB, and/or AURKC inhibitor.

### Definitions

Listed below are definitions of various terms used in this application. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including+5%, 1%, and ±0.1% from the specified value, as such variations are appropriate to perform the disclosed methods.

The term “treat,” “treated,” “treating,” or “treatment” includes the diminishment or alleviation of at least one symptom associated or caused by the state, disorder or disease being treated. In certain embodiments, the treatment comprises bringing into contact with an Aurora kinase an effective amount of a compound of the invention for conditions related to cancers, hemoglobinopathies, or myelodysplastic syndrome.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “inhibit” or “inhibition” means the activity of a targeted protein (e.g. an Aurora kinase) is diminished, and/or the targeted protein (e.g. an Aurora kinase) is degraded.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

The term “targeted protein(s)” is used interchangeably with “target protein(s)”, unless the context clearly dictates otherwise. In one embodiment, a “targeted protein” is AURK. In another embodiment, the targeted protein is a protein complex wherein one protein is an Aurora kinase.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example, in Remington's Pharmaceutical Sciences (Genaro, Ed., Mack Publishing Co., 1985, Easton, Pa.), which is incorporated herein by reference.

This application also encompasses pharmaceutical compositions containing, and methods of treating disorders through administering, pharmaceutically acceptable prodrugs of compounds of the application. For example, compounds of the application having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the application. The amino acid residues include but are not limited to the 20 naturally occurring amino acids commonly designated by three letter symbols and also includes 4-hydroxyproline, hydroxylysine, desmosine, isodemosine, 3-methylhistidine, norvaline, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryl-oxymethyloxy carbonyls, as outlined in Advanced Drug Delivery Reviews, 1996, 19, 1 15. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

An “oral dosage form” includes a unit dosage form prescribed or intended for oral administration.

The terms “AURKA,” “AURKB,” and “AURKC” refer to Aurora kinase-A, Aurora kinase-B, and Aurora kinase-C, respectively, which are serine/threonine kinases linked to tumorigenesis. These kinases are responsible for chromosome segregation, mitotic spindle function, and cytokinesis.

The term “UPP” refers to the Ubiquitin-Proteasome Pathway, which is a critical pathway that regulates proteins and degrades misfolded or abnormal proteins. UPP is central to multiple cellular processes, and if defective or unbalanced, leads to pathogenesis of a variety of diseases. The covalent attachment of ubiquitin to specific protein substrates is achieved through the action of E3 ubiquitin ligases. These ligases comprise over 500 different proteins and are categorized into multiple classes defined by the structural element of their E3 functional activity. For example, cereblon (CRBN) interacts with damaged DNA binding protein 1 and forms an E3 ubiquitin ligase complex with Cullin 4 in which the proteins recognized by CRBN are ubiquitinated and degraded by proteasomes. Various immunomodulatory drugs (IMiDs), e.g., thalidomide and lenalidomide, binds to CRBN and modulates CRBN's role in the ubiquitination and degradation of protein factors involved in maintaining regular cellular function.

Additionally, the compounds of the present application, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “alkyl,” by itself or as part of another substituent means, unless otherwise stated, a straight or branched chain hydrocarbon having the number of carbon atoms designated (i.e., C₁-C₆-alkyl means an alkyl having one to six carbon atoms) and includes straight and branched chains. In an embodiment, C₁-C₆ alkyl groups are provided herein. Examples include methyl, ethyl, propyl, isopropyl, butyl, isobutyl, tert-butyl, pentyl, neopentyl, and hexyl. Other examples of C₁-C₆-alkyl include ethyl, methyl, isopropyl, isobutyl, n-pentyl, and n-hexyl.

The term “alkenyl,” as used herein, denotes a monovalent group derived from a hydrocarbon moiety containing, in certain embodiments, from two to six carbon atoms having at least one carbon-carbon double bond. The double bond may or may not be the point of attachment to another group. Alkenyl groups include, but are not limited to, for example, ethenyl, propenyl, butenyl, 1-methyl-2-buten-1-yl and the like.

As used herein, the term “alkoxy,” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like. In an embodiment, C₁-C₆ alkoxy groups are provided herein.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “aromatic” refers to a carbocycle or heterocycle with one or more polyunsaturated rings and having aromatic character, i.e., having (4n+2) delocalized π (pi) electrons, where n is an integer.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, indenyl and the like. The term “aralkyl,” as used herein, refers to an alkyl residue attached to an aryl ring. Examples include, but are not limited to, benzyl, phenethyl and the like.

As used herein, the term “cycloalkyl” means a non-aromatic carbocyclic system that is partially or fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl. In an embodiment, C₄-C₇ cycloalkyl groups are provided herein.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thiophenyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl. In an embodiment, C₂-C₇ heteroaryl groups are provided herein.

It is to be understood that if an aryl, heteroaryl, cycloalkyl, or heterocycloalkyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thioenyl, and so forth.

The term “heteroaralkyl,” as used herein, refers to an alkyl residue attached to a heteroaryl ring. Examples include, but are not limited to, pyridinylmethyl, pyrimidinylethyl and the like.

The term “heterocyclyl,” or “heterocycloalkyl,” as used herein, refers to a non-aromatic 3-, 4-, 5-, 6- or 7-membered ring or a bi- or tri-cyclic group fused of non-fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, and (iv) the nitrogen heteroatom may optionally be quaternized.

Representative heterocycloalkyl groups include, but are not limited to, [1,3jdioxolane, pyrrolidinvl, pyrazolyl, pyrazolidinyl, imidazolyl, imidazolidinyl, piperidyl, piperazinyl, oxazolidyl, isoxazolidyl, morpholinyl, thiazolidinyl, isothiazolidyl, and tetrahydrofuryl.

The term “alkylamino” refers to a group having the structure —NH(C₁-C₆alkyl), e.g., —NH(C₁-C₆ alkyl), wherein C₁-C₆ alkyl is as previously defined.

The term “dialkylamino” refers to a group having the structure —N(C₁-C₆ alkyl)₂, where C₁-C₆ alkyl is as previously defined.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates. Examples of aliphatic carbonyls include, but are not limited to, acetyl, propionyl, 2-fluoroacetyl, butyryl, 2-hydroxy acetyl, and the like.

In accordance with the application, any of the aryls, substituted aryls, heteroaryls and substituted heteroaryls described herein, can be any aromatic group. Aromatic groups can be substituted or unsubstituted.

As used herein, the term “substituted” means that an atom or group of atoms has replaced hydrogen as the substituent attached to another group.

The term “cancer” includes, but is not limited to, the following cancers: epidermoid oral such as buccal cavity, lip, tongue, mouth, pharynx; cardiac cancers such as sarcoma (angiosarcoma, fibrosarcoma, rhabdomyosarcoma, liposarcoma), myxoma, rhabdomyoma, fibroma, lipoma, and teratoma; lung cancers such as bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatosis hamartoma, mesothelioma; gastrointestinal cancers such as esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Kaposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; genitourinary tract cancers including kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver cancers such as hepatoma (hepatocellular carcinoma), cholangiocarcinoma, hepatoblastoma, angiosarcoma, hepatocellular adenoma, hemangioma, biliary passages; bone cancers such as osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; cancers of the nervous system, including skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); gynecological cancers including uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma. adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; hematologic cancers such as blood (myeloid leukemia (acute and chronic), acute lymphoblastic leukemia, chronic lymphocytic leukemia, myeloproliferative diseases, multiple myeloma, myelodysplasia syndrome), Hodgkin's disease, non-Hodgkin's lymphoma (malignant lymphoma) hairy cell; lymphoid disorders; skin cancers including malignant melanoma, basal cell carcinoma, squamous cell carcinoma, Kaposi's sarcoma, keratoacanthoma, moles dysplastic nevi, lipoma, angioma, dermatofibroma, keloids, psoriasis; cancers of the thyroid gland such as papillary thyroid carcinoma, follicular thyroid carcinoma; medullary' thyroid carcinoma, undifferentiated thyroid cancer, multiple endocrine neoplasia type 2A, multiple endocrine neoplasia type 2B, familial medullary thyroid cancer, pheochromocytoma, paraganglioma; and cancers of the adrenal glands like neuroblastoma. Thus, the term “cancerous cell” as provided herein, includes a cell afflicted by any one of the above-identified conditions.

A “selective AURKB inhibitor” can be identified, for example by comparing the ability of a compound to inhibit AURKB kinase activity to its ability to inhibit the other members of the aurora kinase family or other kinases. For example, a substance may be assayed for its ability to inhibit AURKB kinase activity, as well as AURKA, AURKC and other kinases. In some embodiments the selectivity can be identified by measuring the EC₅₀ or IC₅₀ of the compounds.

In some embodiments, a compound disclosed herein can inhibit an Aurora kinase (e.g., AURKB) by binding to the Aurora kinase, or a protein complexed to Aurora kinase, thus targeting the protein for systematic destruction by the UPP.

### Compounds of the Invention

In an aspect, provided herein are compounds of Formula X:

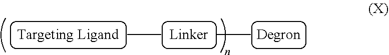

wherein:

the targeting ligand is capable of binding to a targeted protein, such as an aurora kinase (e.g., AURKA, AURKB, or AURKC);

the linker is a group that covalently binds to the targeting ligand and the degron;

the degron is capable of binding to a ubiquitin ligase, such as an E3 ubiquitin ligase (e.g., cereblon); and

n is 1-3.

In an embodiment, n is 1. In another embodiment n is 2. In another embodiment, n is 3.

In another aspect, provided herein are methods of targeting proteins for degradation through the use of compounds, including compounds that link an E3 ubiquitin ligase-binding moiety to a ligand that binds the targeted proteins.

In an embodiment of Formula X, the targeting ligand is a compound of Formula I:

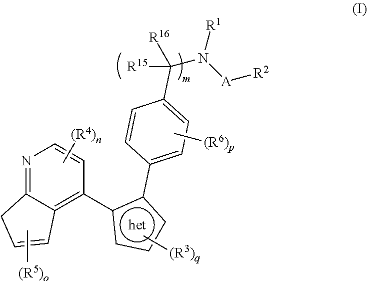

or a pharmaceutically acceptable salt thereof;

wherein:

R¹ is H or C₁-C₆ alkyl;

R² is covalently bonded to the linker and is selected from the group consisting of —NH—, —NCH₃—, —O—, —S—, and —CH₂—;

R³ is selected from the group consisting of C₁-C₆ alkyl, C₁-C₆ alkoxy, hydroxy, amino, and halo;

R⁴ is selected from the group consisting of C₁-C₆ alkyl, C₁-C₆ alkoxy, hydroxy, amino, and halo;

R⁵ is selected from the group consisting of C₁-C₆ alkyl, C₁-C₆ alkoxy, C₅-C₁ heteroaryl, C₆-C₁ aryl, C₃-C₆ cycloalkyl, and C₃-C₆ heterocycloalkyl;

wherein C₅-C₁ heteroaryl and C₆-C₁ aryl are optionally substituted with C₁-C₆ alkyl, C₁-C₆ alkoxy, and C₁-C₆ alkylamine;

R⁶ is selected from the group consisting of C₁-C₆ alkyl, C₁-C₆ alkoxy, hydroxy, amino, and halo;

R¹⁵ is selected from the group consisting of C₁-C₆ alkyl, C₁-C₆ alkoxy, hydroxy, amino, and halo;

R¹⁶ is selected from the group consisting of C₁-C₆ alkyl, C₁-C₆ alkoxy, hydroxy, amino, and halo;

het is C₅-C₁₀ heteroaryl or C₃-C₆ heterocycloalkyl;

A is selected from the group consisting of absent, —C(O)—, —CH₂—, —SO₂—, and —O—;

m is 0, 1, 2, 3, or 4;

n is 0, 1, or 2;

o is 0, 1, or 2;

p is 0, 1, 2, 3, or 4; and

q is 0, 1, 2, or 3;

wherein the targeting ligand is covalently bonded to the linker.

In an embodiment of Formula I, R¹ is methyl. In another embodiment, het is selected from the group consisting of pyrazole, dihydropyrazole, imidazole, pyrrole, and pyrrolidine. In yet another embodiment, het is dihydropyrazole. In still another embodiment, o is 1; and

R⁵ is

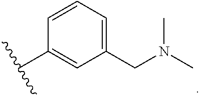 [SMILES: *c1cccc(CN(C)C)c1]

In an embodiment, m is 0; p is 0; q is 0; and n is 0.

In another embodiment, the compound of Formula X is a compound of Formula II:

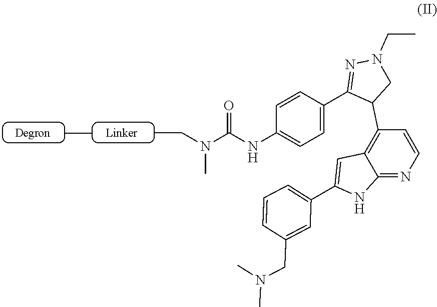

or a pharmaceutically acceptable salt thereof.

In yet another embodiment of Formula X, the linker is selected from the group consisting of

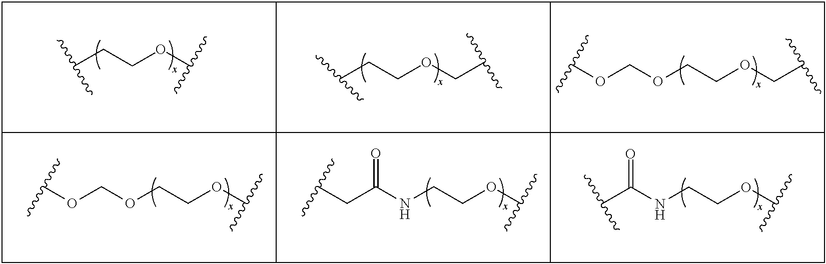

wherein x is 0, 1, 2, 3, 4, or 5; and

the linker is covalently bonded to the degron and covalently bonded to the targeting ligand.

In still another embodiment of Formula X, the linker is C₁-C₆ alkyl, and is covalently bonded to the degron and covalently bonded to the targeting ligand. In an embodiment of Formula X, the linker is C₁-C₆ alkoxy, and is covalently bonded to the degron and covalently bonded to the targeting ligand.

In an embodiment, the compound of Formula X is a compound of Formula L1:

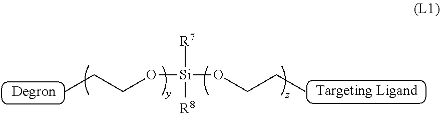

wherein:

R⁷ and R³ are independently selected from the group consisting of methyl, ethyl, propyl, isopropyl, and tert-butyl;

y is 1, 2, 3, 4, or 5;

z is 1, 2, 3, 4, or 5; and

the linker is covalently bonded to the degron and covalently bonded to the targeting ligand.

In an embodiment of Formula X, the targeting ligand is a compound of Formula I, which is capable of binding to a target protein of interest, such AURKs, AURKA, AURKB and/or AURKC.

In another embodiment of Formula X, the degron serves to link a targeted protein, through a linker and a targeting ligand, to a ubiquitin ligase for proteasomal degradation. In one embodiment, the degron is capable of binding to a ubiquitin ligase, such as an E3 ubiquitin ligase. In one embodiment, the degron is capable of binding to cereblon.

In yet another embodiment, the compound of Formula X is a compound of Formula D1:

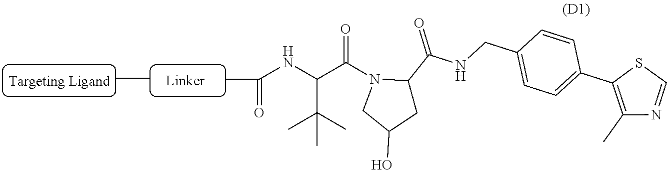

or a stereoisomer thereof;

wherein the degron is covalently bonded to the linker.

In still another embodiment, the compound of Formula X is a compound of Formula D2:

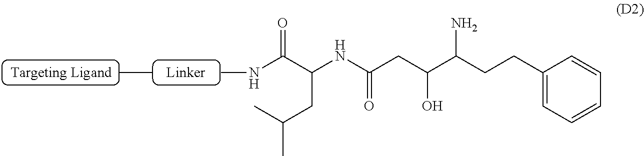

or a stereoisomer thereof;

wherein the degron is covalently bonded to the linker.

In an embodiment, the compound of Formula X is a compound of Formula D3:

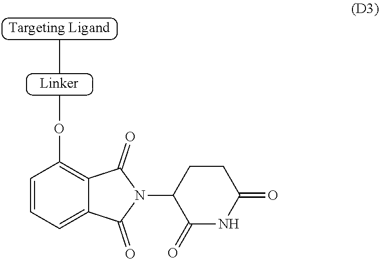

or a stereoisomer thereof;

wherein the degron is covalently bonded to the linker.

In another embodiment, the compound of Formula X is a compound of Formula D4:

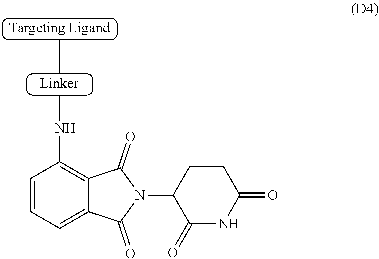

or a stereoisomer thereof;

wherein the degron is covalently bonded to the linker.

In yet another embodiment, the compound of Formula X is a compound of Formula D5:

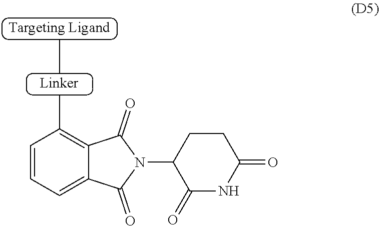

or a stereoisomer thereof;

wherein the degron is covalently bonded to the linker.

In an embodiment, the compound of Formula X is a compound of Formula D6:

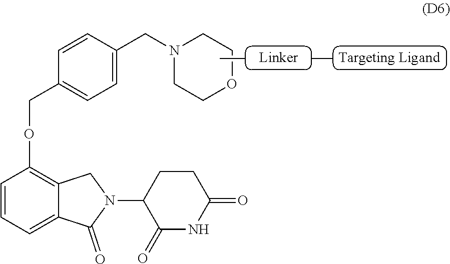

or a stereoisomer thereof;

wherein the degron is covalently bonded to the linker.

In yet another embodiment, the compound of Formula X is a compound of Formula D7:

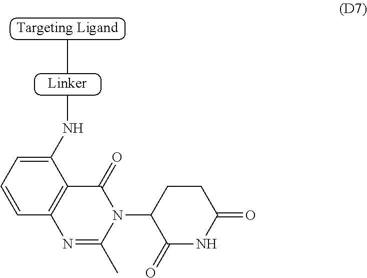

or a stereoisomer thereof;

wherein the degron is covalently bonded to the linker.

In still another embodiment, the compound of Formula X is selected from the group consisting of:

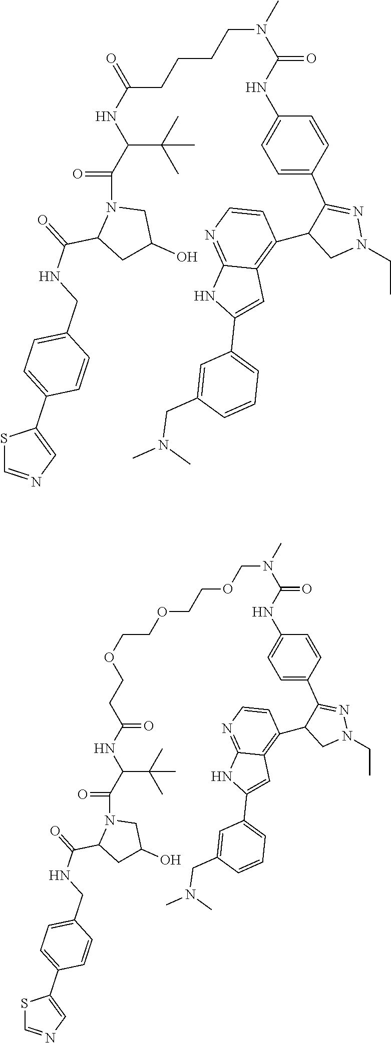 [SMILES: CCN1CC(c2ccnc3[nH]c(-c4cccc(CN(C)C)c4)cc23)C(c2ccc(NC(=O)N(C)CCCCC(=O)NC(C(=O)N3CC(O)CC3C(=O)NCc3ccc(-c4cncs4)cc3)C(C)(C)C)cc2)=N1 ; CCN1CC(c2ccnc3[nH]c(-c4cccc(CN(C)C)c4)cc23)C(c2ccc(NC(=O)N(C)COCCOCCOCCC(=O)NC(C(=O)N3CC(O)CC3C(=O)NCc3ccc(-c4cncs4)cc3)C(C)(C)C)cc2)=N1]

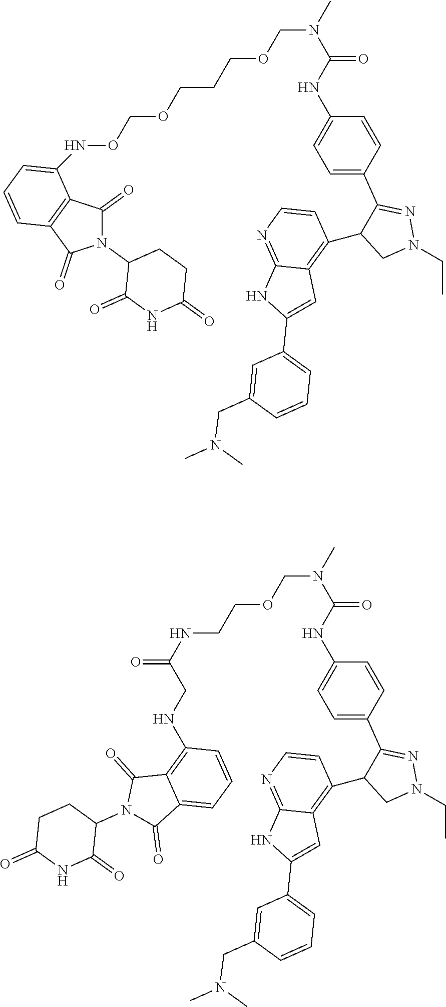 [SMILES: CCN1CC(c2ccnc3[nH]c(-c4cccc(CN(C)C)c4)cc23)C(c2ccc(NC(=O)N(C)COCCCOCONc3cccc4c3C(=O)N(C3CCC(=O)NC3=O)C4=O)cc2)=N1 ; CCN1CC(c2ccnc3[nH]c(-c4cccc(CN(C)C)c4)cc23)C(c2ccc(NC(=O)N(C)COCCNC(=O)CNc3cccc4c3C(=O)N(C3CCC(=O)NC3=O)C4=O)cc2)=N1]

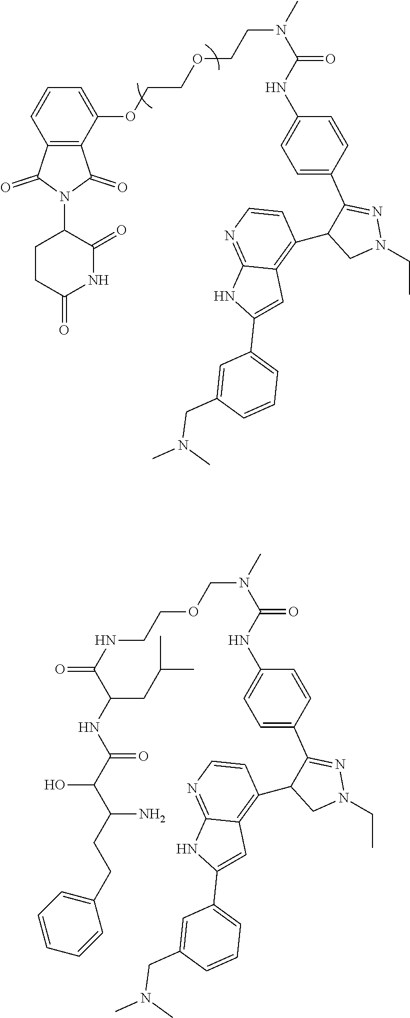

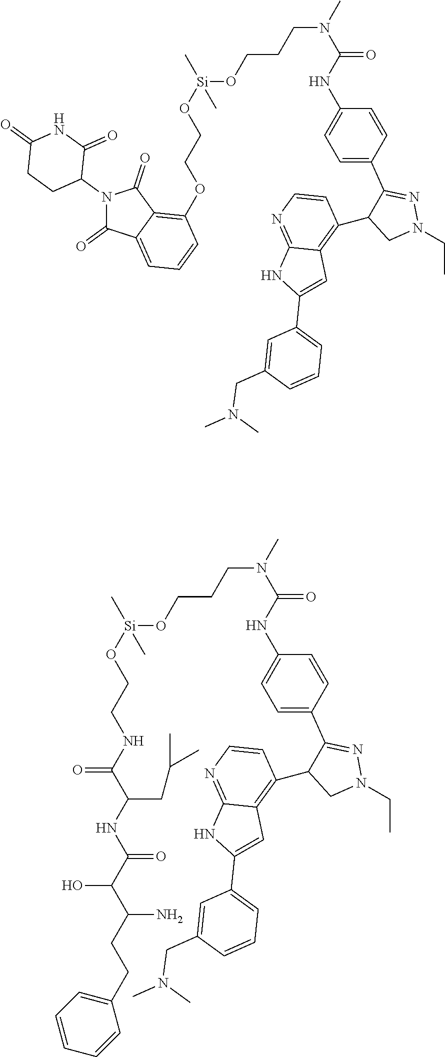 [SMILES: CCN1CC(c2ccnc3[nH]c(-c4cccc(CN(C)C)c4)cc23)C(c2ccc(NC(=O)N(C)CCCO[Si](C)(C)OCCOc3cccc4c3C(=O)N(C3CCC(=O)NC3=O)C4=O)cc2)=N1 ; CCN1CC(c2ccnc3[nH]c(-c4cccc(CN(C)C)c4)cc23)C(c2ccc(NC(=O)N(C)CCCO[Si](C)(C)OCCNC(=O)C(CC(C)C)NC(=O)C(O)C(N)CCc3ccccc3)cc2)=N1]

or pharmaceutically acceptable salts and stereoisomers thereof.

Some of the foregoing compounds can comprise one or more asymmetric centers, and thus can exist in various isomeric forms, e.g., stereoisomers and/or diastereomers.

Accordingly, compounds of the application may be in the form of an individual enantiomer, diastereomer or geometric isomer, or may be in the form of a mixture of stereoisomers. In one embodiment, the compounds of the application are enantiopure compounds. In another embodiment, mixtures of stereoisomers or diastereomers are provided.

Furthermore, certain compounds, as described herein, may have one or more double bonds that can exist as either the Z or E isomer, unless otherwise indicated. The application additionally encompasses the compounds as individual Z/E isomers substantially free of other E/Z isomers and alternatively, as mixtures of various isomers.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In another embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, the compounds described herein include a ²H (i.e., deuterium) isotope.

In still another embodiment, substitution with positron emitting isotopes, such as ¹¹C, ¹⁸F, ¹⁵O and ¹³N, is useful in Positron Emission Topography (PET) studies for examining ligand binding. Isotopically-labeled compounds are prepared by any suitable method or by processes using an appropriate isotopically-labeled reagent in place of the non-labeled reagent otherwise employed.

The specific compounds described herein, and other compounds encompassed by one or more of the formulas described herein having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as described herein are modified by the use of appropriate reagents and conditions, for the introduction of the various moieties found in the Formulas as provided herein.

Compounds described herein are synthesized using any suitable procedures starting from compounds that are available from commercial sources, or are prepared using procedures described herein.

### Methods of Treatment

In another aspect, provided herein are methods of inhibiting a kinase in a subject in need thereof, comprising administering to the subject an effective amount of a compound of Formula X, or a pharmaceutically acceptable salt thereof, or a pharmaceutical composition comprising a compound of Formula X, or a pharmaceutically acceptable salt thereof. In an embodiment, the kinase is AURKA. In another embodiment, the kinase is AURKB. In yet another embodiment, the kinase is AURKC.

In yet another aspect, provided herein are methods of modulating (e.g., decreasing) the amount of a kinase in a subject in need thereof, comprising administering to the subject a therapeutically effective amount of a compound of Formula X, or a pharmaceutically acceptable salt thereof, or a pharmaceutical composition comprising a compound of Formula X, or a pharmaceutically acceptable salt thereof.

In still another aspect, provided herein are methods of treating a disease in a subject in need thereof, comprising administering to the subject an effective amount of a compound of Formula X or a pharmaceutically acceptable salt thereof, or a pharmaceutical composition comprising a compound of Formula X, or a pharmaceutically acceptable salt thereof. In an embodiment, the disease is a proliferative disease.

In other embodiments, the disease or disorder is inflammation, arthritis, rheumatoid arthritis, spondyloarthropathies, gouty arthritis, osteoarthritis, juvenile arthritis, and other arthritic conditions, systemic lupus erythematosus (SLE), skin-related conditions, psoriasis, eczema, burns, dermatitis, neuroinflammation, allergy, pain, neuropathic pain, fever, pulmonary disorders, lung inflammation, adult respiratory distress syndrome, pulmonary sarcoidosis, asthma, silicosis, chronic pulmonary inflammatory disease, and chronic obstructive pulmonary disease (COPD), cardiovascular disease, arteriosclerosis, myocardial infarction (including post-myocardial infarction indications), thrombosis, congestive heart failure, cardiac reperfusion injury, as well as complications associated with hypertension and/or heart failure such as vascular organ damage, restenosis, cardiomyopathy, stroke including ischemic and hemorrhagic stroke, reperfusion injury, renal reperfusion injury, ischemia including stroke and brain ischemia, and ischemia resulting from cardiac/coronary bypass, neurodegenerative disorders, liver disease and nephritis, gastrointestinal conditions, inflammatory bowel disease, Crohn's disease, gastritis, irritable bowel syndrome, ulcerative colitis, ulcerative diseases, gastric ulcers, viral and bacterial infections, sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, herpes virus, myalgias due to infection, influenza, autoimmune disease, graft vs. host reaction and allograft rejections, treatment of bone resorption diseases, osteoporosis, multiple sclerosis, cancer, leukemia, lymphoma, colorectal cancer, brain cancer, bone cancer, epithelial call-derived neoplasia (epithelial carcinoma), basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovarian cancer, cervical cancer, lung cancer, breast cancer, skin cancer, squamous cell and/or basal cell cancers, prostate cancer, renal cell carcinoma, and other known cancers that affect epithelial cells throughout the body, chronic myelogenous leukemia (CML), acute myeloid leukemia (AML) and acute promyelocytic leukemia (APL), angiogenesis including neoplasia, metastasis, central nervous system disorders, central nervous system disorders having an inflammatory or apoptotic component, Alzheimer's disease, Parkinson's disease, Huntington's disease, amyotrophic lateral sclerosis, spinal cord injury, and peripheral neuropathy, or B-Cell Lymphoma.

In yet another aspect, provided herein is a method of treating a kinase-mediated disorder, the method comprising administering to a subject in need thereof an effective amount of a pharmaceutical composition comprising a compound disclosed herein, or a pharmaceutically acceptable salt thereof and a pharmaceutically acceptable carrier. In some embodiments, the compound binds to AURKB. In another embodiment, the compound binds to AURKC. In still another embodiment, the compound binds to AURKA. In other embodiments, the subject is administered an additional therapeutic agent. In other embodiments, the pharmaceutical composition comprising a compound and the additional therapeutic agent are administered simultaneously or sequentially.

In an aspect, provided herein are methods of treating cancer in a subject in need thereof, comprising administering to the subject an effective amount of a compound of Formula X or a pharmaceutically acceptable salt thereof, or a pharmaceutical composition comprising a compound of Formula X, or a pharmaceutically acceptable salt thereof. In another embodiment, the subject is identified as being in need of AURKA, AURKB, and/or AURKC inhibition for the treatment of cancer. In an embodiment, the cancer is the cancer is selected from the group consisting of chronic lymphocytic leukemia (CML), acute lymphoblastic leukemia (ALL), acute myelogenous leukemia (AML), peripheral T-cell lymphoma (PTCL), myelodysplastic syndrome (MDS), diffuse large B-cell lymphoma, and Hodgkin's lymphoma. In another embodiment, the cancer is selected from the group consisting of non-small cell lung carcinoma (NSCLC), mesothelioma, glioblastoma, oral cancer, malignant endometrium, hepatocellular carcinoma, testicular germ cell tumors, ovarian cancer, thyroid cancer, colon cancer, and prostate cancer. In yet another embodiment, the cancer is selected from the group consisting of lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In still another embodiment, the cancer is selected from the group consisting of epidermoid oral cancers, cardiac cancers, lung cancers, gastrointestinal cancers, pancreatic cancers, cancers of the small bowel or small intestines, cancers of the large bowel or large intestines, genitourinary tract cancers, liver cancers, bone cancers, cancers of the nervous system, gynecological cancers, breast cancers, hematologic cancers, and cancers of the lymphoid.

In one embodiment, the compounds provided herein target proteins, such as AURKA, AURKB, and/or AURKC for degradation, which have numerous advantages over inhibitors of protein function (e.g., kinase activity) and can a) overcome resistance in certain cases; b) prolong the kinetics of drug effect by destroying the protein, thus requiring resynthesis of the protein even after the compound has been metabolized; c) target all functions of a protein at once rather than a specific catalytic activity or binding event; d) expand the number of drug targets by including all proteins that a ligand can be developed for, rather than proteins whose activity (e.g., kinase activity) can be affected by a small molecule inhibitor, antagonist or agonist; and e) have increased potency compared to inhibitors due to the possibility of the small molecule acting catalytically.

In an embodiment, the compounds provided herein are capable of producing a degradation or loss of 30% to 100% of the target protein. In another embodiment, the compounds provided herein relate to the loss of 50-100% of the target protein. In yet another embodiment, the compounds provided herein relate to the loss of 75-95% of the targeted protein.

In another aspect, provided herein are compounds capable of modulating (e.g., decreasing) the amount of a targeted protein (e.g., AURKB). In an embodiment, the compounds are capable of degrading a targeted protein (e.g., AURKB) through the UPP pathway.

Modulation of AURKA, AURKB, and/or AURKC through UPP-mediated degradation by a compound provided herein provides a novel approach to the treatment, prevention, or amelioration of diseases or disorders in which AURKA, AURKB, and/or AURKC plays a role, including but not limited to, cancer and metastasis, inflammation, arthritis, systemic lupus erythematosus, skin-related disorders, pulmonary' disorders, cardiovascular disease, ischemia, neurodegenerative disorders, liver disease, gastrointestinal disorders, viral and bacterial infections, central nervous system disorders, Alzheimer's disease, Parkinson's disease, Huntington's disease, amyotrophic lateral sclerosis, spinal cord injury, and peripheral neuropathy. Further, modulation of AURKA, AURKB, and/or AURKC through UPP-mediated degradation by a compound provided herein also provides a new paradigm for treating, preventing, or ameliorating diseases or disorders in which AURKA, AURKB, and/or AURKC is deregulated.

In an embodiment, the compounds provided herein are more efficacious in treating a disease or condition (e.g., cancer) than, or is capable of treating a disease or condition resistant to, the targeting ligand, when the targeting ligand is administered alone (i.e., not bonded to a linker and a degron). In another embodiment, the compounds provided herein are capable of modulating (e.g., decreasing) the amount of AURKA, AURKB, and/or AURKC, and thus are useful in treating a disease or condition (e.g., cancer) in which AURKA, AURKB, and/or AURKC plays a role.

In another embodiment, a compound provided herein that is more efficacious in treating a disease or condition than, or is capable of treating a disease or condition resistant to, the targeting ligand, when the targeting ligand is administered alone (i.e., not bonded to a linker and a degron), is more potent in inhibiting the growth of cells (e.g., cancer cells) or decreasing the viability of cells (e.g., cancer cells), than the targeting ligand, when the targeting ligand is administered alone (i.e., not bonded to a linker and a degron).

In some embodiments, the inhibition of AURKA, AURKB, and/or AURKC activity is measured by IC₅₀. In some embodiments, the inhibition of AURKA, AURKB, and/or AURKC activity is measured by EC₅₀. In some embodiments, the inhibition of and/or binding to AURKA, AURKB, and/or AURKC activity is shown by measuring Kₐ and K_{d}.

Potency of the inhibitor can be determined by EC₅₀ value. A compound with a lower EC₅₀ value, as determined under substantially similar conditions, is a more potent inhibitor relative to a compound with a higher EC₅₀ value. In some embodiments, the substantially similar conditions comprise determining a AURKB-dependent phosphorylation level, in vitro or in vivo (e.g., in cells expressing a wild-type AURKB, a mutant AURKB, or a fragment of any thereof).

Potency of the inhibitor can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining a AURKB-dependent phosphorylation level, in vitro or in vivo (e.g., in cells expressing a wild-type AURKB, a mutant AURKB, or a fragment of any thereof).

In some embodiments, a compound disclosed herein can diminish the activity of a target protein or result in the degradation of the target protein based on the measured Kₐ and K_{d} values of the compound.

In some embodiments, a compound disclosed herein can diminish the activity of a target protein or result in the degradation of the target protein based on the compound's ability to tag the targeted protein for degradation by the UPP.

In some embodiments, the compounds of the present application are selective over other kinases. As used herein, “selective”, “selective AURKB inhibitor”, or “selective AURKB compound” refers to a compound, e.g., a compound of Formula X, that effectively inhibits AURKB kinase to a greater extent than any other kinase enzyme, particularly any enzyme from the aurora kinase family (e.g., AURKA, AURKB, AURKC etc.).

In certain embodiments, the compounds provided herein are AURKB inhibitors that exhibit at least 2-fold, 3-fold, 5-fold, 10-fold, 25-fold, 50-fold or 100-fold selectivity over other kinases (e.g., AURKA, AURKB, AURKC etc.). In various embodiments, the compounds of the application exhibit 1000-fold selectivity over other kinases.

In certain embodiments, the compounds of the application are AURKB inhibitors that exhibit at least 2-fold, 3-fold, 5-fold, 10-fold, 25-fold, 50-fold or 100-fold selectivity over other cyclin-dependent kinases (e.g., AURKA, AURKB, AURKC etc.). In various embodiments, the compounds of the application exhibit 1000-fold selectivity over other cyclin-dependent kinases.

In another aspect, the application provides a method of modulating a kinase, comprising contacting the kinase with a compound disclosed herein, or a pharmaceutically acceptable salt thereof, or with a pharmaceutical composition disclosed herein. In some embodiments, the kinase is AURKB. In another embodiment, the kinase is AURKA. In still another embodiment, the kinase is AURKC.

In still another aspect, provided herein is a method of treating or preventing a proliferative disease, comprising administering to a subject in need thereof an effective amount of a compound disclosed herein, or a pharmaceutically acceptable salt thereof.

In another aspect, provided herein is a method of treating cancer, comprising administering to a subject in need thereof an effective amount of a pharmaceutical composition comprising a compound disclosed herein, or a pharmaceutically acceptable salt thereof and a pharmaceutically acceptable carrier.

In yet another aspect, provided herein is a method of treating cancer in a subject, wherein the subject is identified as being in need of AURKB inhibition for the treatment of cancer, comprising administering to the subject an effective amount of a compound disclosed herein, or a pharmaceutically acceptable salt thereof.

In still another aspect, provided herein is a method of treating cancer in a subject, wherein the subject is identified as being in need of AURKB inhibition for the treatment of cancer, comprising administering to the subject an effective amount of a pharmaceutical composition comprising a compound disclosed herein, or a pharmaceutically acceptable salt thereof and a pharmaceutically acceptable carrier. In another embodiment, the subject is identified as being in need of AURKA inhibition. In still another embodiment, the subject is identified as being in need of AURKC inhibition.

In an embodiment of the methods, the subject is a human.

In another aspect, the application provides a pharmaceutical composition comprising a compound disclosed herein, or a pharmaceutically acceptable salt thereof and a pharmaceutically acceptable carrier, for use in the manufacture of a medicament for treating or preventing a disease in which Aurora kinase plays a role. In an embodiment, the Aurora kinase is AURKA. In another embodiment, the Aurora kinase is AURKB. In yet another embodiment, the Aurora kinase is AURKC.

In still another aspect, the application provides a pharmaceutical composition comprising a compound disclosed herein, or a pharmaceutically acceptable salt thereof and a pharmaceutically acceptable carrier, for use in treating or preventing a disease in which AURKB plays a role.

As inhibitors of AURKA, AURKB, and/or AURKC, the compounds and compositions disclosed herein are particularly useful for treating or lessening the severity of a disease, condition, or disorder where a protein kinase is implicated in the disease, condition, or disorder. In one aspect, the present application provides a method for treating or lessening the severity of a disease, condition, or disorder where a protein kinase is implicated in the disease state. In another aspect, this application provides a method for treating or lessening the severity of a disease, condition, or disorder with functional compounds that inhibit enzymatic activity by binding to the protein kinase. Another aspect provides a method for treating or lessening the severity of a kinase disease, condition, or disorder by inhibiting enzymatic activity of the kinase with a protein kinase inhibitor.

In some embodiments, said method is used to treat or prevent a condition selected from autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, said condition is selected from a proliferative disorder and a neurodegenerative disorder.

In an aspect, provided herein is a method of treating or preventing cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intraepithelial tissue.

In as aspect, provided herein is a method of treating neurodegenerative diseases in a subject in need thereof, comprising administering to the subject a therapeutically effective amount of a compound disclosed herein. In an embodiment, the neurodegenerative disease is selected from Adrenoleukody strophy (ALD), Alexander's disease, Alper's disease, Alzheimer's disease, Amyotrophic lateral sclerosis (Lou Gehrig's Disease), Ataxia telangiectasia. Batten disease (Spielmeyer-Vogt-Sjogren-Batten disease), Bovine spongiform encephalopathy (BSE), Canavan disease, Cockayne syndrome, Corticobasal degeneration, Creutzfeldt-Jakob disease, Familial fatal insomnia, Frontotemporal lobar degeneration, Huntington's disease, HI-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease. Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis, and Toxic encephalopathy.

As inhibitors of AURKA, AURKB, and/or AURKC, the compounds and compositions disclosed herein are also useful in biological samples. One aspect of the application relates to inhibiting protein kinase activity in a biological sample, which method comprises contacting said biological sample with a functional compound of the application or a composition comprising said functional compound. The term “biological sample”, as used herein, means an in vitro or an ex vivo sample, including, without limitation, cell cultures or extracts thereof; biopsied material obtained from a mammal or extracts thereof; and blood, saliva, urine, feces, semen, tears, or other body fluids or extracts thereof. Inhibition of protein kinase activity in a biological sample is useful for a variety of purposes that are known to one of skill in the art. Examples of such purposes include, but are not limited to, blood transfusion, organ-transplantation, and biological specimen storage.

Another aspect of this application relates to the study of AURKA, AURKB, and/or AURKC in biological and pathological phenomena; the study of intracellular signal transduction pathways mediated by such protein kinases; and the comparative evaluation of new protein kinase inhibitors.

Examples of such uses include, but are not limited to, biological assays such as enzyme assays, cell-based assays, and protein binding assays. The activity of the compounds and compositions of the present application as Aurora kinase inhibitors and binders may be assayed in vitro, in vivo, or in a cell line. In vitro assays include assays that determine either inhibition of the kinase activity or degradation of the kinase. Alternate in vitro assays quantitate the ability of the compound to bind to the protein kinase and may be measured either by radio labelling the inhibitor prior to binding, isolating the inhibitor/kinase complex and determining the amount of radio label bound, or by running a competition experiment where new inhibitors are incubated with the kinase bound to known radioligands. Detailed conditions for assaying a compound utilized in this application as an inhibitor or binder of various kinases are set forth in the Examples below.

### Kits

In an aspect, provided herein are kits comprising a compound of Formula X capable of inhibiting kinase activity. In an embodiment, the kinase is Aurora kinase-A (AURKA). In another embodiment, the kinase is Aurora kinase-B (AURKB). In yet another embodiment, the kinase is Aurora kinase-C (AURKC).

In yet another aspect, provided herein are kits comprising a compound of Formula X capable of modulating (e.g., decreasing) the amount of AURKA, AURKB, and/or AURKC.

In an aspect, provided herein are kits for the treatment of a disease, wherein the kit comprises a compound of Formula X. In an embodiment, the disease is a kinase-mediated disease. In another embodiment, the kinase is AURKA. In yet another embodiment, the kinase is AURKB. In another embodiment, the kinase is AURKC. In still another embodiment, the disease is a proliferative disease. In an embodiment, the proliferative disease is cancer.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### Administration/Dosage/Formulations

In another aspect, provided herein is a pharmaceutical composition comprising at least one compound of the invention, together with a pharmaceutically acceptable carrier.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the invention are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds of the invention are formulated using one or more pharmaceutically acceptable excipients or carriers. In one embodiment, the pharmaceutical compositions of the invention comprise a therapeutically effective amount of a disclosed compound and a pharmaceutically acceptable carrier.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

Compounds and compositions of the application can be administered in therapeutically effective amounts in a combinational therapy with one or more therapeutic agents (pharmaceutical combinations) or modalities, e.g., an anti-proliferative, anti-cancer, immunomodulatory or anti-inflammatory agent. Where the compounds of the application are administered in conjunction with other therapies, dosages of the co-administered compounds will of course vary depending on the type of co-drug employed, on the specific drug employed, on the condition being treated and so forth. Compounds and compositions of the application can be administered in therapeutically effective amounts in a combinational therapy with one or more therapeutic agents (pharmaceutical combinations) or modalities, e.g., anti-proliferative, anti-cancer, immunomodulatory or anti-inflammatory agent, and/or non-drug therapies, etc. For example, synergistic effects can occur with anti-proliferative, anti-cancer, immunomodulatory or anti-inflammatory substances. Where the compounds of the application are administered in conjunction with other therapies, dosages of the co-administered compounds will of course vary depending on the type of co-drug employed, on the specific drug employed, on the condition being treated and so forth.

Combination therapy includes the administration of the subject compounds in further combination with one or more other biologically active ingredients (such as, but not limited to, a second AURKB inhibitor, a second and different antineoplastic agent, aurora kinase inhibitor (i.e., AURKA, AURKB, AURKC etc.) and non-drug therapies (such as, but not limited to, surgery or radiation treatment). For instance, the compounds of the application can be used in combination with other pharmaceutically active compounds, preferably compounds that are able to enhance the effect of the compounds of the application. The compounds of the application can be administered simultaneously (as a single preparation or separate preparation) or sequentially to the other drug therapy or treatment modality. In general, a combination therapy envisions administration of two or more drugs during a single cycle or course of therapy.

In another aspect of the application, the compounds may be administered in combination with one or more separate pharmaceutical agents, e.g., a chemotherapeutic agent, an immunotherapeutic agent, or an adjunctive therapeutic agent. In an embodiment, the separate pharmaceutical agent is selected from the group consisting of a PARP inhibitor, an HDAC inhibitor, an EGFR inhibitor, a HER2/NEU inhibitor, platinum, taxanes, an SRC inhibitor, a PI3K inhibitor, an AKT inhibitor, a JAK-2 inhibitor, an mTOR inhibitor, and bleomycin.

The following examples further illustrate aspects of the present invention. However, they are in no way a limitation of the teachings or disclosure of the present invention as set forth.

### EXAMPLES

The invention is further illustrated by the following examples, which should not be construed as further limiting. The practice of the present invention will employ, unless otherwise indicated, conventional techniques of organic synthesis, cell biology, cell culture, molecular biology, transgenic biology, microbiology and immunology, which are within the skill of the art.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### Synthetic Procedures

Compounds of the present application can be prepared by following the steps outlined in Scheme 1 and Scheme 2, which comprise different sequences of assembling intermediates. Starting materials are either commercially available or made by known procedures in the reported literature or as illustrated.

The general method for preparing compounds of the present application using intermediates is outlined below. The targeting ligand can be coupled to the linker under standard conditions well understood in the art of organic synthesis. The targeting ligand-linker intermediate can be further functionalized at the linker's terminus with the degron under typical coupling conditions.

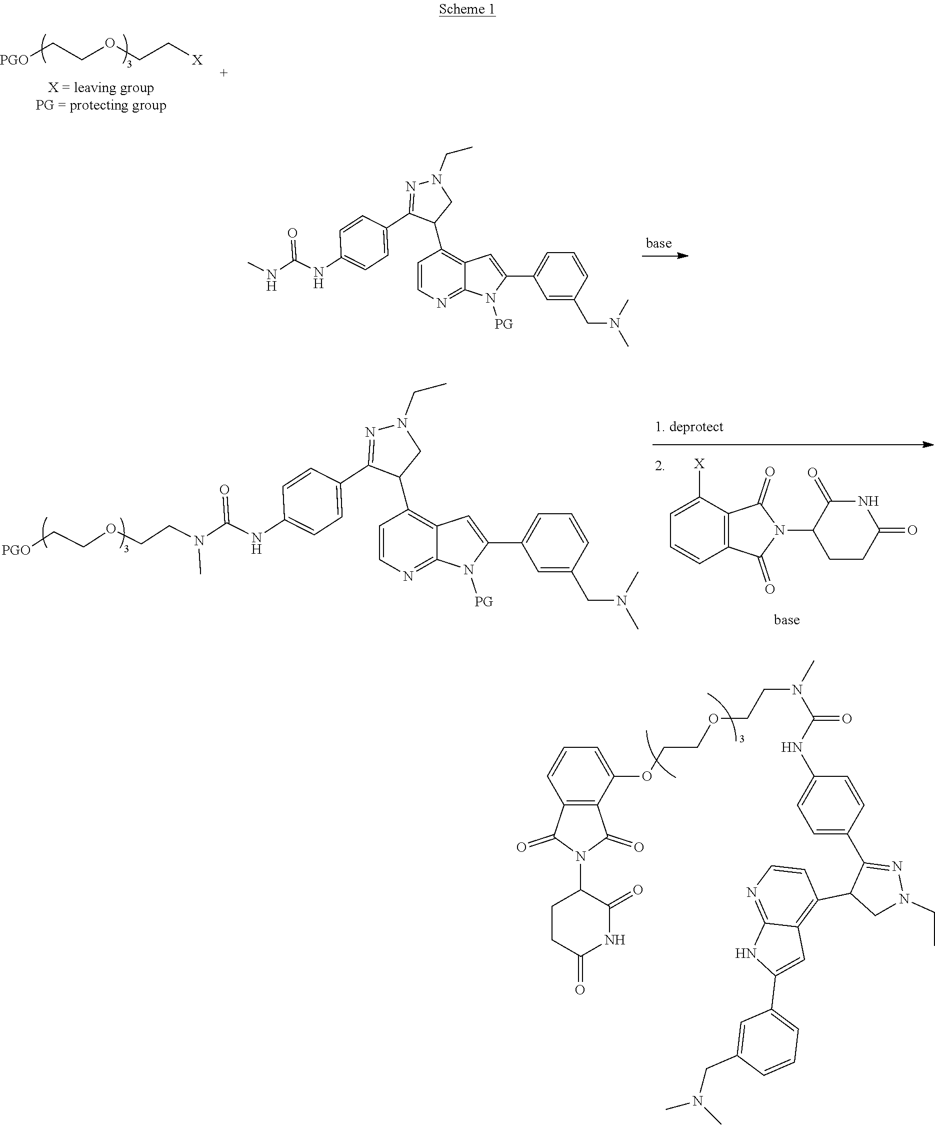

Alternatively, a general method for preparing the compounds disclosed herein is shown in Scheme 2, which changes the order of addition.

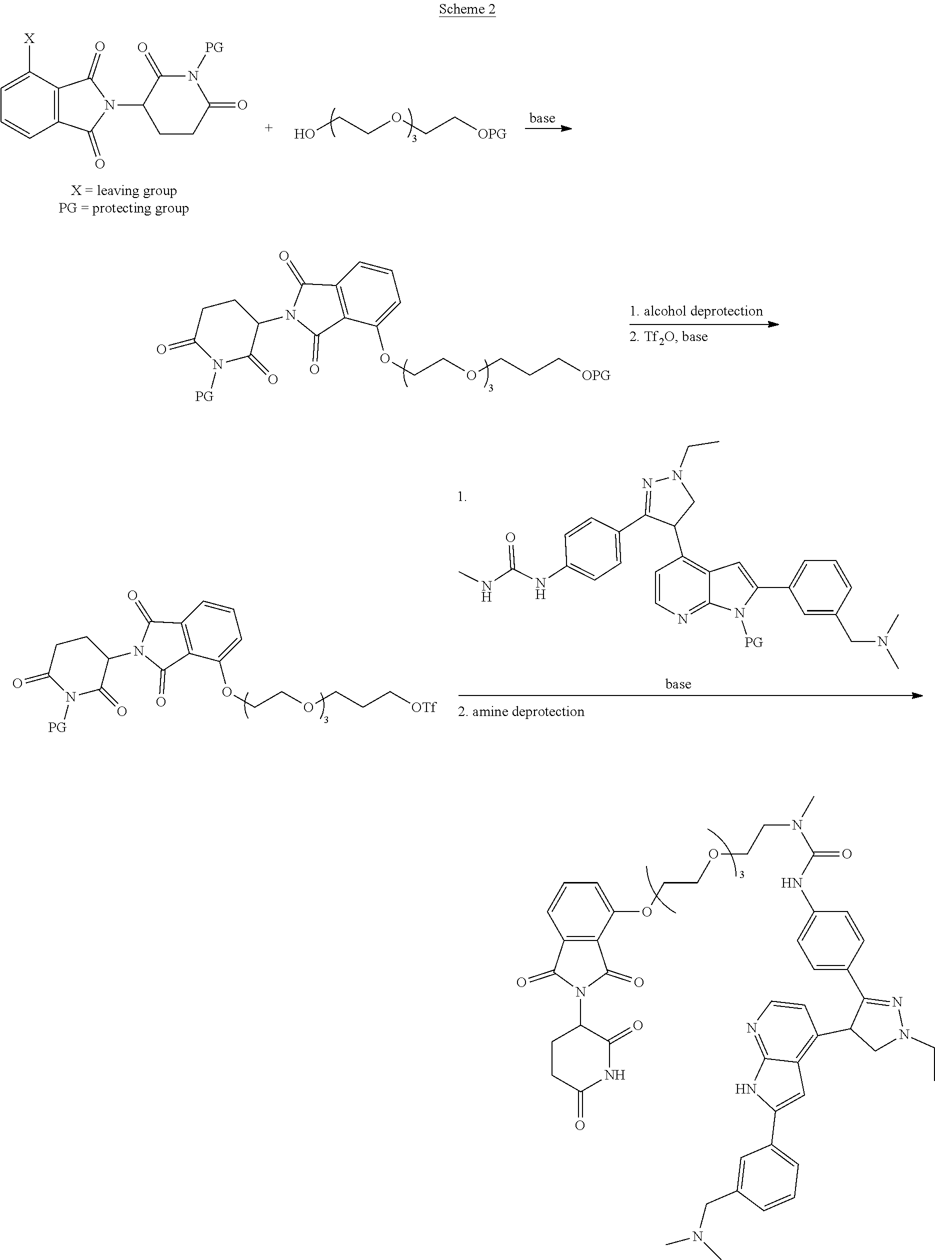

The degron and linker can be coupled under typical S_{N}AR conditions. Conversion of the linker's terminus into a good leaving group, e.g., a triflate, allows for a second coupling reaction under basic conditions between the targeting ligand and the linker. Subsequent deprotection results in a compound of Formula X. These synthetic procedures are standard in the art of organic synthesis and are easily understood by the skilled artisan.

### Biological Assays

### Cell Viability Assay

Wild-type or cereblon null cells are treated with various concentrations of a compound provided herein and allowed to grow. Cells are then assayed to determine cell viability by measuring the amount of ATP present which is an indicator of cell metabolic activity. Results are graphed as relative luminescent values.

### Enzyme Degradation Assay

Cells are treated with a control or a compound provided herein alone or in combination with an agent that blocks proteasomal degradation at a single concentration or various concentrations. After treatment, cells are washed and harvested by resuspending in buffer and lysed on ice 30 minutes. Lysates are then cleared by centrifugation. Samples are boiled and equal amount of protein is loaded onto polyacrylamide gel. The gel is transferred to nitrocellulose and blotted for AURKA, AURKB, AURKC H3-Ser10 phosphorylation or Tubulin.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound of Formula X: 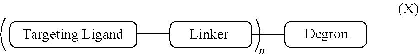
or a pharmaceutically acceptable salt thereof;
wherein:
the targeting ligand is a ligand capable of binding to a targeted protein;
the linker is a group that covalently binds to the targeting ligand and the degron;
the degron is capable of binding to a ubiquitin ligase; and
n is 1-3.

2. The compound of claim 1, wherein the targeting ligand is a compound of Formula I: 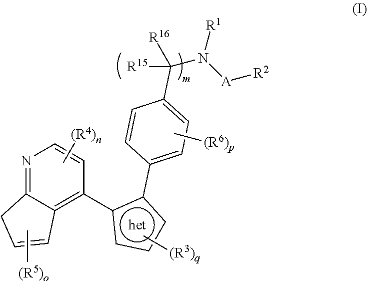
or a pharmaceutically acceptable salt thereof;
wherein:
R¹ is H or C₁-C₆ alkyl;
R² is covalently bonded to the linker and is selected from the group consisting of a direct bond, —NH—, —NCH₃—, —O—, —S—, and —CH₂—;
R³ is selected from the group consisting of C₁-C₆ alkyl, C₁-C₆ alkoxy, hydroxy, amino, and halo;
R⁴ is selected from the group consisting of C₁-C₆ alkyl, C₁-C₆ alkoxy, hydroxy, amino, and halo;
R⁵ is selected from the group consisting of C₁-C₆ alkyl, C₁-C₆ alkoxy, C₅-C₁ heteroaryl, C₆-C₁₀ aryl, C₃-C₆ cycloalkyl, and C₃-C₆ heterocycloalkyl;
wherein C₅-C₁ heteroaryl and C₆-C₁ aryl are optionally substituted with C₁-C₆ alkyl, C₁-C₆ alkoxy, and C₁-C₆ alkylamine;
R⁶ is selected from the group consisting of C₁-C₆ alkyl, C₁-C₆ alkoxy, hydroxy, amino, and halo;
R¹⁵ is selected from the group consisting of C₁-C₆ alkyl, C₁-C₆ alkoxy, hydroxy, amino, and halo;
R¹⁶ is selected from the group consisting of C₁-C₆ alkyl, C₁-C₆ alkoxy, hydroxy, amino, and halo;
het is C₅-C₁ₐ heteroaryl or C₃-C₆ heterocycloalkyl;
A is selected from the group consisting of absent, —C(O)—, —CH₂—, —SO₂—, and —O—;
m is 0, 1, 2, 3, or 4;
n is 0, 1, or 2;
is 0, 1, or 2;
p is 0, 1, 2, 3, or 4; and
q is 0, 1, 2, or 3.

3. The compound of claim 2, wherein R¹ is methyl.

4. The compound of claim 2, wherein het is selected from the group consisting of pyrazole, dihydropyrazole, imidazole, pyrrole, and pyrrolidine.

5. (canceled)

6. The compound of claim 2, wherein o is 1; and
R⁵ is

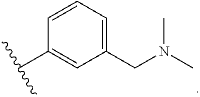 [SMILES: *c1cccc(CN(C)C)c1]

7. The compound of claim 2, wherein
m is 0;
p is 0;
q is 0; and
n is 0.

8. The compound of claim 1, wherein the linker is selected from the group consisting of 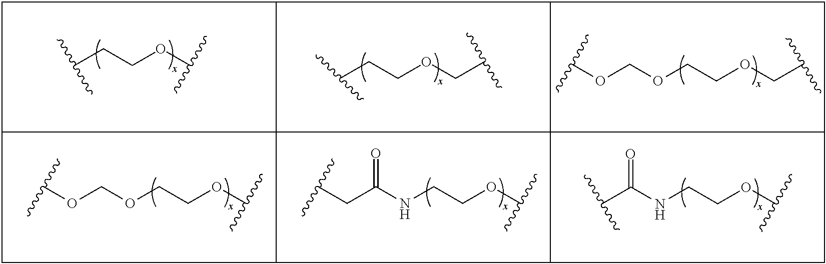
wherein x is 0, 1, 2, 3, 4, or 5; and
the linker is covalently bonded to the degron and covalently bonded to the targeting ligand.

9. The compound of claim 1, wherein the linker is C₁-C₆ alkyl, and is covalently bonded to the degron and covalently bonded to the targeting ligand.

10. The compound of claim 1, wherein the compound of Formula X is a compound of Formula L1: 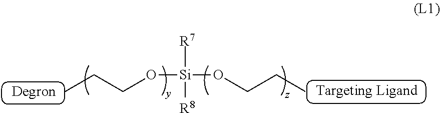
wherein:
R⁷ and R⁸ are independently selected from the group consisting of methyl, ethyl, propyl, isopropyl, and tert-butyl;
y is 1, 2, 3, 4, or 5;
z is 1, 2, 3, 4, or 5; and
the linker is covalently bonded to the degron and covalently bonded to the targeting ligand.

11. The compound of claim 1, wherein the compound of Formula X is a compound of Formula D1: 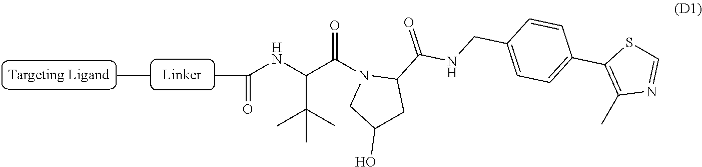
or a stereoisomer thereof;
wherein the degron is covalently bonded to the linker.

12. The compound of claim 1, wherein the compound of Formula X is a compound of Formula D2: 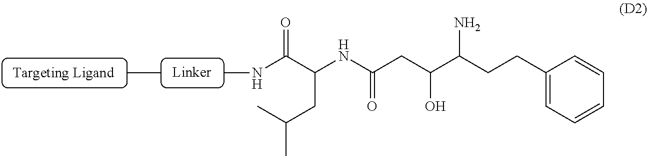
or a stereoisomer thereof;
wherein the degron is covalently bonded to the linker.

13. The compound of claim 1, wherein the compound of Formula X is a compound of Formula D3: 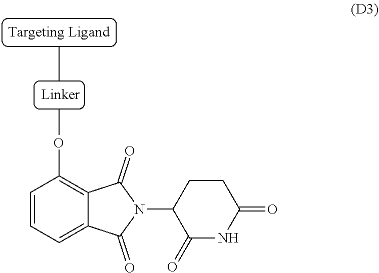
or a stereoisomer thereof;
wherein the degron is covalently bonded to the linker.

14. The compound of claim 1, wherein the compound of Formula X is a compound of Formula D4: 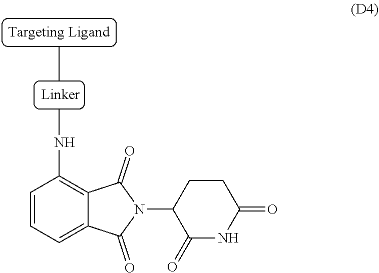
or a stereoisomer thereof;
wherein the degron is covalently bonded to the linker.

15. The compound of claim 1, wherein the compound of Formula X is a compound of Formula D5: 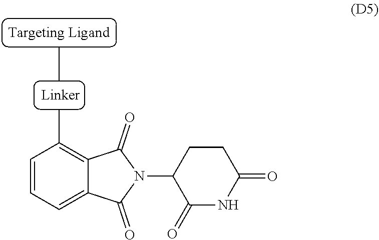
or a stereoisomer thereof;
wherein the degron is covalently bonded to the linker.

16. The compound of claim 1, wherein the compound of Formula X is a compound of Formula D6: 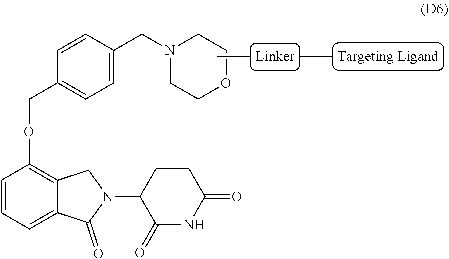
or a stereoisomer thereof;
wherein the degron is covalently bonded to the linker.

17. The compound of claim 1, wherein the compound of Formula X is a compound of Formula D7: 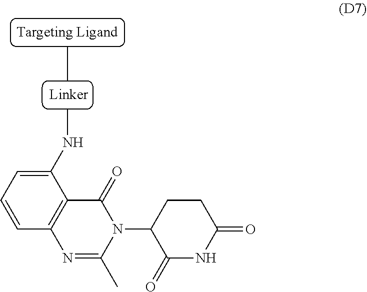
or a stereoisomer thereof;
wherein the degron is covalently bonded to the linker.

18. The compound of claim 1, wherein the compound of Formula X is a compound of Formula II: 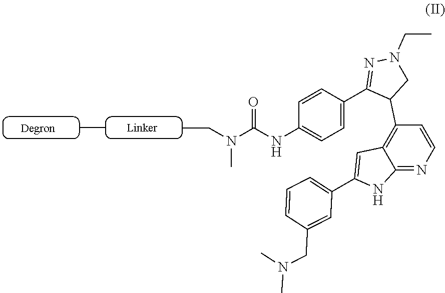
or a pharmaceutically acceptable salt thereof;
wherein the targeting ligand is covalently bonded to the linker.

19. The compound of claim 1, wherein the compound of Formula X is selected from the group consisting of 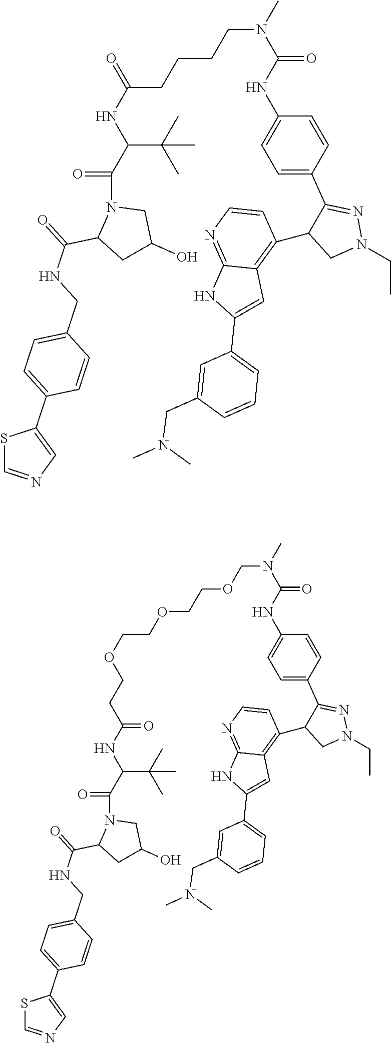 [SMILES: CCN1CC(c2ccnc3[nH]c(-c4cccc(CN(C)C)c4)cc23)C(c2ccc(NC(=O)N(C)CCCCC(=O)NC(C(=O)N3CC(O)CC3C(=O)NCc3ccc(-c4cncs4)cc3)C(C)(C)C)cc2)=N1 ; CCN1CC(c2ccnc3[nH]c(-c4cccc(CN(C)C)c4)cc23)C(c2ccc(NC(=O)N(C)COCCOCCOCCC(=O)NC(C(=O)N3CC(O)CC3C(=O)NCc3ccc(-c4cncs4)cc3)C(C)(C)C)cc2)=N1] 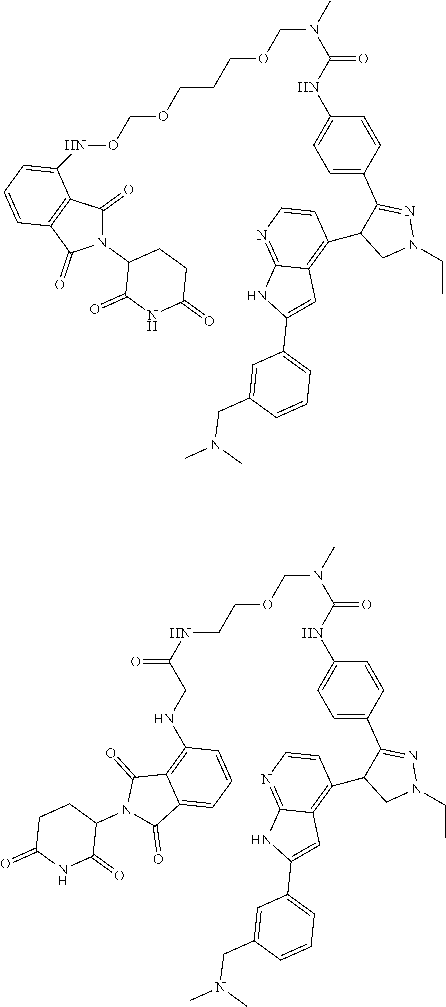 [SMILES: CCN1CC(c2ccnc3[nH]c(-c4cccc(CN(C)C)c4)cc23)C(c2ccc(NC(=O)N(C)COCCCOCONc3cccc4c3C(=O)N(C3CCC(=O)NC3=O)C4=O)cc2)=N1 ; CCN1CC(c2ccnc3[nH]c(-c4cccc(CN(C)C)c4)cc23)C(c2ccc(NC(=O)N(C)COCCNC(=O)CNc3cccc4c3C(=O)N(C3CCC(=O)NC3=O)C4=O)cc2)=N1] 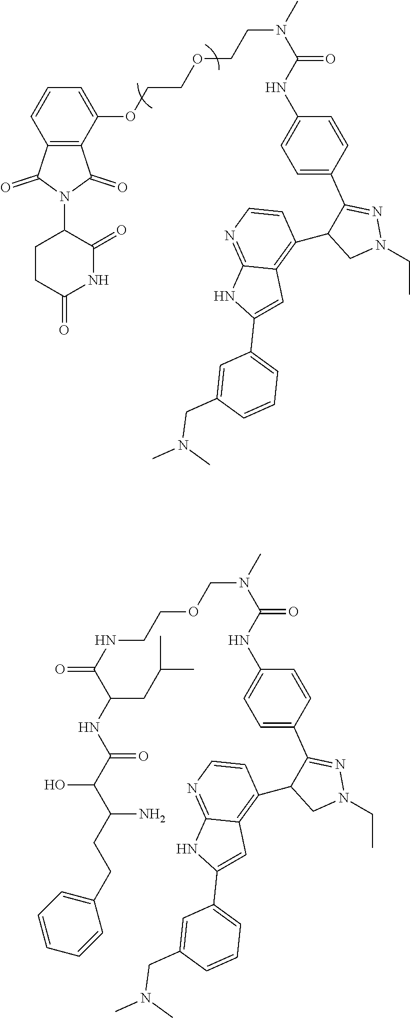 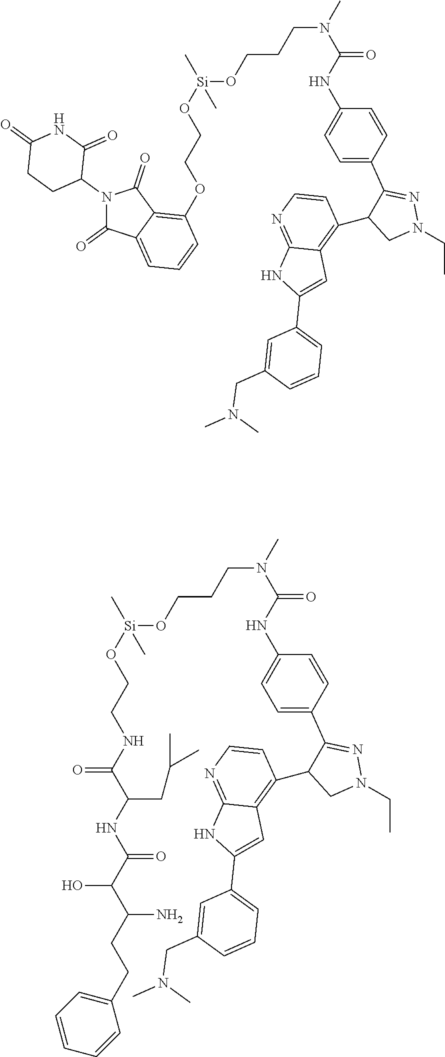 [SMILES: CCN1CC(c2ccnc3[nH]c(-c4cccc(CN(C)C)c4)cc23)C(c2ccc(NC(=O)N(C)CCCO[Si](C)(C)OCCOc3cccc4c3C(=O)N(C3CCC(=O)NC3=O)C4=O)cc2)=N1 ; CCN1CC(c2ccnc3[nH]c(-c4cccc(CN(C)C)c4)cc23)C(c2ccc(NC(=O)N(C)CCCO[Si](C)(C)OCCNC(=O)C(CC(C)C)NC(=O)C(O)C(N)CCc3ccccc3)cc2)=N1]
or pharmaceutically acceptable salts and stereoisomers thereof.

20. A pharmaceutical composition comprising a compound of claim 1, or a pharmaceutically acceptable salt thereof, together with a pharmaceutically acceptable carrier.

21. A method of inhibiting Aurora kinase in a subject in need thereof, comprising administering to the subject an effective amount of a compound of claim 1.

22. (canceled)

23. The method of claim 21, wherein the kinase is Aurora kinase-A (AURKA), Aurora kinase-B (AURKB), or Aurora kinase-C (AURKC).

24-25. (canceled)

26. A method of modulating the amount of Aurora kinase in a subject in need thereof, comprising administering to the subject an effective amount of a compound of claim 1.

27-28. (canceled)

29. The method of claim 26, wherein the kinase is Aurora kinase-A (AURKA), Aurora kinase-B (AURKB), or Aurora kinase-C (AURKC).

30-37. (canceled)

38. A method of treating cancer in a subject in need thereof, comprising administering to the subject an effective amount of a compound of claim 1.

39-41. (canceled)

42. The method of claim 38, wherein the cancer is selected from the group consisting of non-small cell lung carcinoma (NSCLC), mesothelioma, glioblastoma, oral cancer, malignant endometrium, hepatocellular carcinoma, testicular germ cell tumors, ovarian cancer, thyroid cancer, colon cancer, prostate cancer, chronic lymphocytic leukemia (CML), acute lymphoblastic leukemia (ALL), acute myelogenous leukemia (AML), peripheral T-cell lymphoma (PTCL), myelodysplastic syndrome (MDS), diffuse large B-cell lymphoma, and Hodgkin's lymphoma.

43. (canceled)
